(19) United States
(12) Patent Application Publication
(10) Pub. No.: US 2015/0312739 A1
(43) Pub. Date: Oct. 29, 2015

(54) COMPUTER PROGRAM, METHOD, AND SYSTEM FOR OBTAINING AND PROVIDING EMERGENCY SERVICES

(71) Applicant: Shauncey J. Burt, Jackson, MS (US)

(72) Inventor: Shauncey J. Burt, Jackson, MS (US)

(21) Appl. No.: 14/261,998

(22) Filed: Apr. 25, 2014

(51) Int. Cl. H04W 4/22; A61B 5/021; A61B 7/00; A61B 5/00; A61B 5/11

(52) U.S. Cl. CPC H04W 4/22; A61B 5/7282; A61B 5/7264; A61B 5/747; A61B 5/0022; A61B 5/1112; A61B 5/0077; A61B 7/00; A61B 5/746; A61B 5/686; A61B 5/021; A61B 5/6898

(57) ABSTRACT

A computer program, method, and system for obtaining and providing emergency services, which includes an initial step of receiving user profile data from a plurality of emergency service users. A next step includes presenting a solicitation for a civilian user to select an emergency event type. A next step includes receiving information indicative of the emergency event type. Next, the emergency event type is compared with and the user profile data of the emergency service users, and a notification is sent to a first emergency service user of either a first type or a second type. A next step includes presenting a solicitation for the first emergency service user to communicate with the civilian user. Upon receiving information indicative of an assent to communicate with the civilian user, a communication link is opened between the first emergency service user and the civilian user.

### FIELD

Embodiments of the present invention provide a computer program, method, and system for obtaining and providing emergency services. In more detail, embodiments of the present invention are directed to a computer program, method, and system for facilitating the ability of a user undergoing an emergency event to obtain emergency services from an emergency service user. Embodiments additionally facilitate the emergency service user's ability to provide emergency services.

### BACKGROUND

Presently-available technology for requesting emergency services has been in place for several decades. Such technology generally comprises a telecommunications operator that can be contacted via a land-line or cellular network by an individual undergoing an emergency event. Upon the operator being contacted, the operator must obtain information from the individual before emergency services can be provided to the individual. Such information is generally obtained by the operator asking a plurality of questions of the individual. However, obtaining information by asking numerous questions can be time-consuming and can often lead to inaccuracies and mistakes being made.

Similarly, once the operator has obtained the necessary information from the individual undergoing the emergency event, the operator must provide such information to an appropriate emergency service provider. Such information is generally provided to the emergency services provider from the operator via direct communication, such as through a radio-frequency network or telecommunications network. Nevertheless, as previously mentioned, providing information via direct communication can be time-consuming and can lead to inaccuracies and mistakes being made.

Such deficiencies in obtaining and providing information can be detrimental to the individual undergoing the emergency event. Specifically, any time losses and inaccuracies will reduce the efficiency and the effectiveness of the emergency services provided by emergency service providers. Furthermore, any reduction in the efficiency and effectiveness of emergency services provided to an individual undergoing an emergency event can significantly increasing the likelihood of damages caused to the individual by the emergency event. As such, there is a need for a computer program, method, and system that facilitates the ability to obtain and provide emergency services efficiently and effectively.

### SUMMARY

Embodiments of the present invention provide for a computer program, method, and system directed to obtaining emergency services. In more detail, embodiments of the present invention include a method comprising the steps of receiving user profile data from a computing device of a primary user and receiving information indicative of the primary user reporting an event. A next step includes receiving primary event data from the computing device of the primary user. Thereafter, in a next step, embodiments provide for the primary event data to be available for presentation on a computing device of a secondary user. A next step includes generating, based on the user profile data of the primary user, an event classification used to classify event data as being associated with the event. Next, a plurality of user profile data is received from computing devices of a plurality of tertiary users, and a next step includes receiving a plurality of tertiary event data from the computing devices of the plurality of tertiary users. Thereafter, a next step includes classifying, based on the user profile data of the tertiary users, a first portion of the tertiary event data as being within the event classification. A final step includes providing for the first portion of the tertiary event data to be available for presentation on the computing device of the secondary user, and a remaining portion of the tertiary event data not included within the first portion to not be available for presentation on the computing device of the secondary user.

Embodiments include an additional method of organizing data from a plurality of users comprising the initial step of receiving user profile data from a computing device of a first user. A next step includes receiving event data from the computing device of the first user and storing the event data in a database. Next, an event description is received from the computing device of the first user. A next step includes generating, based on the user profile data or the event description, a set of one or more event data tags used to identify the event data stored in the database. A next step includes receiving an indication of a second user performing a query of the database using a search term. Next, a determination is made as to whether the search term matches any of the one or more event data tags. Finally, upon determining that the search term matches any of the one or more event data tags, present the event data on the computing device of the second user.

Embodiments of the present invention include a further method for generating a communication link between users, with the method comprising the initial step of receiving user profile data from a plurality of emergency service users, and with the user profile data indicating whether each emergency service user in the plurality is an emergency service user of a first type or a second type. A next step includes providing a set of computer-executable instructions that, when executed by the civilian user's computing device, generates a user interface displayable on an electronic display of the computing device. A next step includes presenting, via the user interface of the civilian user's computing device, a solicitation for the civilian user to select an emergency event type. Next, information indicative of the emergency event type is received. Thereafter, in the next step, based on a comparison between the emergency event type and the user profile data of the emergency service users, a notification is sent to an emergency service user of either the first type or the second type, with the emergency service user to which the notification is sent being a first emergency service user. A next step includes providing a set of computer-executable instructions that, when executed by a computing device of the first emergency service user, generates a user interface displayable on an electronic display coupled to the first emergency service user's computing device. A next step includes presenting, via the user interface of the first emergency service user's computing device, a solicitation for the first emergency service user to communicate with the civilian user. In a final step, upon receiving information indicative of an assent to communicate with the civilian user, a communication link is opened between the first emergency service user and the civilian user, via the respective computing devices of the first emergency service user and the civilian user.

This summary is provided to introduce a selection of concepts in a simplified form that are further described below in the detailed description. This summary is not intended to identify key features or essential features of the claimed subject matter, nor is it intended to be used to limit the scope of the claimed subject matter. Other aspects and advantages of the present invention will be apparent from the following detailed description of the embodiments and the accompanying drawing figures.

### BRIEF DESCRIPTION OF THE DRAWING FIGURES

Embodiments of the present invention are described in detail below with reference to the attached drawing figures, wherein:

FIG. 1 is a flow chart of a method for generating a communication link between a civilian user and one or more emergency service users according to embodiments of the present invention;

FIG. 2 is a schematic depiction of a system for obtaining and providing emergency services in accordance with embodiments of the present invention;

FIG. 3 is an illustration of graphical user interface displayed on a computing device for obtaining personal information user profile data according to embodiments of the present invention;

FIG. 4 is an additional illustration of graphical user interface displayed on a computing device for obtaining personal information user profile data according to embodiments of the present invention;

FIG. 5 is an illustration of graphical user interface displayed on a computing device for obtaining health information user profile data according to embodiments of the present invention;

FIG. 6 is an illustration of graphical user interface displayed on a computing device for obtaining residential information user profile data according to embodiments of the present invention;

FIG. 7 is an illustration of graphical user interface displayed on a computing device for obtaining vehicle information user profile data according to embodiments of the present invention;

FIG. 8 is an illustration of graphical user interface displayed on a computing device for requesting emergency services by a user undergoing an emergency event;

FIG. 9 is an illustration of graphical user interface displayed on a computing device for a user to provide an event description of an emergency event;

FIG. 10 is an illustration of graphical user interface displayed on a computing device for an emergency service user to assent to provide emergency services to a civilian user undergoing an emergency event;

FIG. 11 is an illustration of graphical user interface displayed on a computing device for presenting summary information related to the emergency event;

FIG. 12 is a flow chart of a method capturing data associated with an emergency event from a plurality of users according to embodiments of the present invention; and

FIG. 13 is a flow chart of a method for organizing data associated with an emergency event received from a plurality of users according to embodiments of the present invention.

The drawing figures do not limit the present invention to the specific embodiments disclosed and described herein. The drawings are not necessarily to scale, emphasis instead being placed upon clearly illustrating the principles of the invention.

### DETAILED DESCRIPTION OF THE EMBODIMENTS

The following detailed description of the invention references the accompanying drawings that illustrate specific embodiments in which the invention can be practiced. The embodiments are intended to describe aspects of the invention in sufficient detail to enable those skilled in the art to practice the invention. Other embodiments can be utilized and changes can be made without departing from the scope of the present invention. The following detailed description is, therefore, not to be taken in a limiting sense. The scope of the present invention is defined only by the appended claims, along with the full scope of equivalents to which such claims are entitled.

In this description, references to “one embodiment,” “an embodiment,” or “embodiments” mean that the feature or features being referred to are included in at least one embodiment of the technology. Separate references to “one embodiment,” “an embodiment,” or “embodiments” in this description do not necessarily refer to the same embodiment and are also not mutually exclusive unless so stated and/or except as will be readily apparent to those skilled in the art from the description. For example, a feature, structure, act, etc. described in one embodiment may also be included in other embodiments, but is not necessarily included. Thus, the present technology can include a variety of combinations and/or integrations of the embodiments described herein.

The computer program, method, and system of embodiments of the present invention are operable to provide an electronic resource, such as an online application or a mobile application, which allows a first user to obtain a plurality of emergency services and which allows a second user to provide a plurality of emergency services. As a non-limiting example, embodiments of the present invention can be implemented by an individual undergoing an emergency event, such as a criminal emergency, a fire emergency, a medical emergency or the like. Such an individual can implement embodiments of the present invention via a computing device so as to obtain emergency services from emergency service personnel who can assist the individual undergoing the emergency event. Similarly, emergency service personnel can implement embodiments of the present invention via a computing device to facilitate the ability to provide emergency services to such an individual.

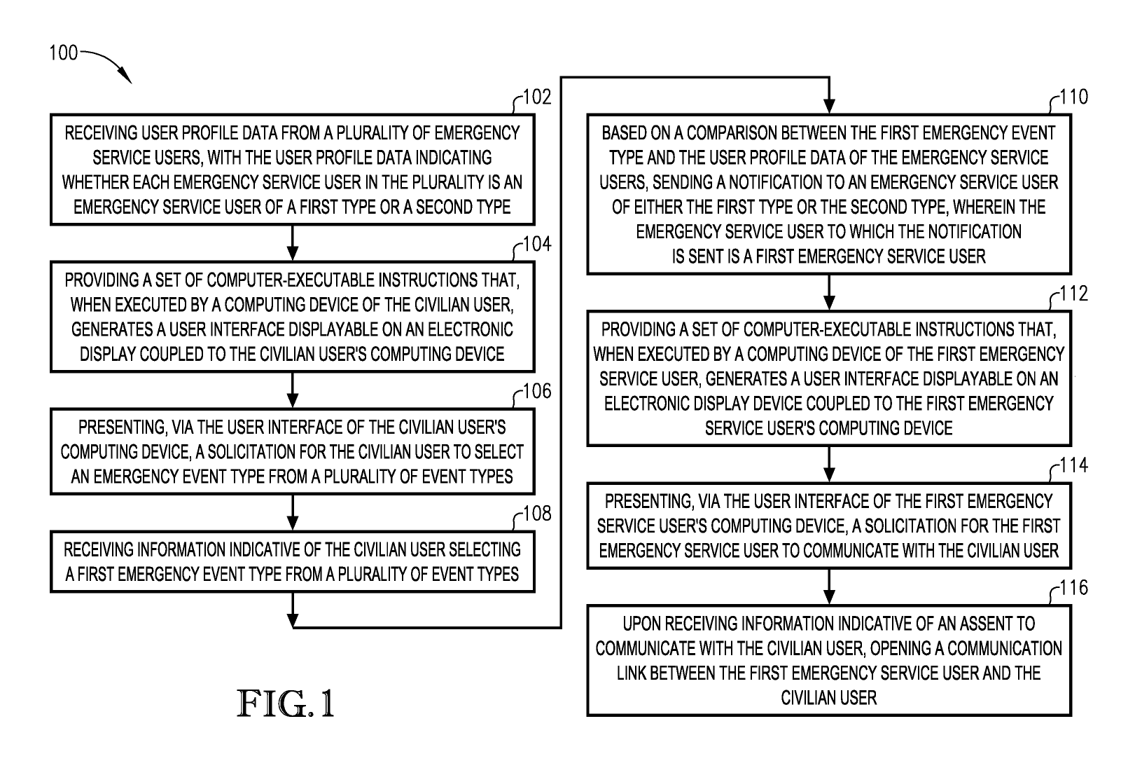

For example, embodiments of the present invention are operable for generating a communication link between a civilian user and one or more types of emergency service users (e.g., police, fire, EMT, etc.). In particular, and with reference to FIG. 1, embodiments include a method 100 with an initial Step 102 of receiving user profile data from a plurality of emergency service users, wherein the user profile data indicates whether each emergency service user in the plurality is an emergency service user of a first type or a second type. A next Step 104 includes providing a set of computer-executable instructions that, when executed by the civilian user's computing device, generates a user interface displayable on an electronic display of the computing device. Next Step 106 includes presenting, via the user interface of the civilian user's computing device, a solicitation for the civilian user to select an emergency event type. In Step 108, embodiments provide for receiving information indicative of the emergency event type. Thereafter, in Step 110, based on a comparison between the emergency event type and the user profile data of the emergency service users, embodiments send a notification to an emergency service user of either the first type or the second type, wherein the emergency service user to which the notification is sent is a first emergency service user. Step 112 includes providing a set of computer-executable instructions that, when executed by a computing device of the first emergency service user, generates a user interface displayable on an electronic display coupled to the first emergency service user's computing device. Next Step 114 includes presenting, via the user interface of the first emergency service user's computing device, a solicitation for the first emergency service user to communicate with the civilian user. In final Step 116, upon receiving information indicative of an assent to communicate with the civilian user, a communication link is opened between the first emergency service user and the civilian user, via the respective computing devices of the first emergency service user and the civilian user.

The computer program of embodiments of the present invention comprises a plurality of code segments executable by a computing device for performing the steps of the method of the present invention. The steps of the method may be performed in the order shown in FIG. 1, or they may be performed in a different order. Furthermore, some steps may be performed concurrently as opposed to sequentially. Also, some steps may be optional.

### System Description

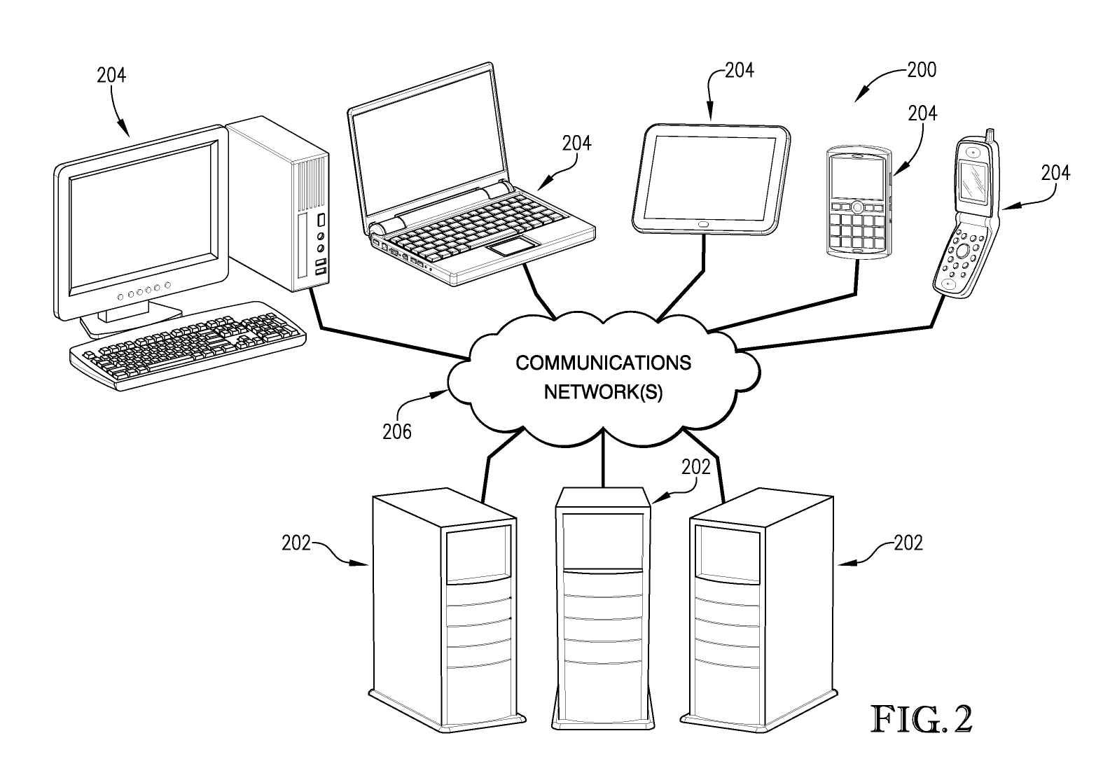

The computer program, system, and method of embodiments of the present invention may be implemented in hardware, software, firmware, or combinations thereof using the emergency services system 200, shown in FIG. 2, which broadly comprises server devices 202, computing devices 204, and a communications network 206. The server devices 202 may include computing devices that provide access to one or more general computing resources, such as database services, file server services, Internet services, application server services, electronic mail services, data transfer services, and the like. In particular, the server devices 202 may provide access to an emergency services database (or a plurality of databases) that stores information and data necessary for the implementation of various functions and features of and for the general operability of embodiments of the present invention.

The server devices 202 and the computing devices 204 may include any device, component, or equipment with a processing element and associated memory elements. The processing element may implement operating systems, and may be capable of executing the computer program, which is also generally known as instructions, commands, software code, executables, applications, apps, and the like. The processing element may include processors, microprocessors, microcontrollers, field programmable gate arrays, and the like, or combinations thereof. The memory elements may be capable of storing or retaining the computer program and may also store data, typically binary data, including text, databases, graphics, audio, video, combinations thereof, and the like. The memory elements may also be known as a “computer-readable storage medium” and may include random access memory (RAM), read only memory (ROM), flash drive memory, floppy disks, hard disk drives, optical storage media such as compact discs (CDs or CDROMs), digital video disc (DVD), Blu-Ray™, and the like, or combinations thereof. In addition to these memory elements, the server devices 202 may further include file stores comprising a plurality of hard disk drives, network attached storage, or a separate storage network, such as a “cloud” storage network.

The computing devices 204 may specifically include mobile communication devices (including wireless devices), smart-phones, tablets, laptop computers, desktop computers, work stations, and the like, or combinations thereof. In preferred embodiments, the computing device 204 will have an electronic display, such as a cathode ray tube (CRT), liquid crystal display (LCD), plasma (PDP), light emitting diode (LED or OLED), touch screen (capacitive/resistive), or combinations thereof, which are operable to display visual graphics, images, text, etc. In certain embodiments, the computer program of the present invention facilitates interaction and communication through a graphical user interface (GUI) that is generated by the processing element and displayed via the electronic display. The GUI enables the user to interact with the electronic display by touching or point at display areas to provide information to a user control interface, which is discussed in more detail below. In additional preferred embodiments, the computing device 204 may include an image capturing device such as a digital camera, video camera, optical canner, or the like, such that the computing device is operable to capture, store, and transmit digital images and/or videos. In further preferred embodiments, the computing device will include a global positioning system (GPS) receiver for obtaining signals from GPS satellites. From the obtained signals, the processing element of the computing device 204 is operable to determine a geospatial location (geo-location) of the computing device. As will be discussed in more detail below, embodiments of the present invention provide for a determination of the computing device's geo-location through other transceiver components, such as an RF component, WiFi component, Bluetooth™ component, or the like.

The computing devices 204 may also include a user control interface that enables one or more users to share information and commands with the computing devices or server devices 202. The user interface may comprise one or more functionable inputs such as buttons, keyboard, switches, scrolls wheels, voice recognition elements such as a microphone, pointing devices such as mice, touchpads, tracking balls, styluses. The user control interface may also include a speaker for providing audible instructions and feedback. Further, the user control interface may comprise wired or wireless data transfer elements, such as a Bluetooth™ communication component, removable memory, data transceivers, and/or transmitters, to enable the user and/or other computing devices to remotely interface with the computing device 204.

The communications network 206 may be wired or wireless (e.g., RF, WiFi, Bluetooth™, etc.) and may include servers, routers, switches, wireless receivers and transmitters, and the like, as well as electrically conductive cables or optical cables. The communications network 206 may also include local, metro, or wide area networks, as well as the Internet, or other cloud networks. Furthermore, the communications network 206 may include cellular or mobile phone networks, as well as landline phone networks, public switched telephone networks, fiber optic networks, or the like.

Both the server devices 202 and the computing devices 204 may be connected to the communications network 206. Server devices 202 may be operable to communicate with other server devices 202 or with computing devices 204 through the communications network 206. Likewise, computing devices 204 may be operable to communicate with other computing devices 204 or with server devices 202 through the communications network 206. As described, the connection to the communications network 206 may be wired or wireless. Thus, the server devices 202 and the computing devices 204 may include the appropriate components to establish a wired or a wireless connection. Furthermore, embodiments of the present invention provide for the encryption of all communications transmitted between server devices 202, between computing devices 204, and between server devices and computing devices. As such, the server devices 202 and the computing devices 204 may include the appropriate components necessary to establish such encryption (e.g., AES 24, 32, 64, or 128-bit encryption).

In certain embodiments, the computing devices 204 may be capable of communicating over any available cellular phone network, regardless of whether the computing device is registered with a particular cellular network service provider. Such embodiments are particularly important because the computing devices 204 are implemented to obtain and provide emergency services to/for users undergoing an emergency event, as described in detail below. As such, in certain instances in which the computing device 204 is out of cellular range of its registered cellular network service provider, embodiments of the present invention provide for the computing device to connect to any other cellular network available via any other service provider. In some embodiments, to facilitate such capabilities, the computing device 204 may be configured to emit an emergency code, which instructs all cellular network providers that the computing device 204 is being used in an emergency event and, thus, requires access to any available cellular network.

The computer program of the present invention may run on one or more server devices 202 or, alternatively, may run on one or more computing devices 204. In further alternatives, a first portion of the program, code, or instructions may execute on a first server device 202 or a first computing device 204, while a second portion of the program, code, or instructions may execute on a second server device 202 or a second computing device 204. In some embodiments, other portions of the program, code, or instructions may execute on other server devices 202 as well. For example, information related to the operation of embodiments of the present invention may be stored on a memory element associated with the server device 202, such that the information is remotely accessible to users of the computer program via one or more computing devices 204. Alternatively, the information related to the operation of embodiments of the present invention may be directly stored on the memory element associated with the one or more computing devices 204 of the user. In additional embodiments of the present invention, a portion of the information related to the operation of embodiments of the present invention may be stored on the server device 202, while another portion may be stored on the one or more computing devices 204. As such, the various actions, functions, and calculations described herein as being performed by or using the computer program may actually be performed by one or more computers, processors, or other computational devices, such as the computing devices 204 and/or server devices 202, independently or cooperatively executing portions of the computer program. In certain embodiments, the computer program will facilitate interaction with other computer programs, software, firmware, or the like via one or more application programming interfaces (APIs). As such, certain functions and features described below as being performed via embodiments of the present invention may be performed via external pieces of software (e.g., Google Voice™, Google Translate™, Google Maps™, etc.) that are connected to the embodiments of the present invention via an API.

In certain embodiments of the present invention, the computer program may be embodied in a stand-alone program downloaded on a user's computing device 204 or in a web-accessible program made available by the server device 202 and that is accessible by the user's computing device 204 via the network 206. Regardless, either the stand-alone program or the web-accessible program (i.e., the electronic resource) permits the user to access embodiments of the present invention. For the stand-alone program, a downloadable version of the computer program may be stored, at least in part, on the server device 202. A user can download at least a portion of the computer program onto the computing device 204 via the network 206. In such embodiments of the present invention, the computer program may be an “application,” such as an “app” for a mobile device. After the computer program has been downloaded, the program can be installed on the computing device 204 in an executable format. For the web-accessible computer program, the user may simply access the computer program via the communications network 206 (e.g., the Internet) with the computing device 204.

Once the user has access to the electronic resource, via the computer program installed on a user's computing device 204 or the web, certain embodiments may provide for users to create accounts with which to access the electronic resource. The user accounts may be stored within the memory elements of the server device 202 (or in the associated database) or within the memory elements of the computing device 204. Certain embodiments of the present invention may provide for at least three types of user accounts, including a civilian account, an emergency services account (ES account), and an administrator account (i.e., an “admin account”). Each user account may provide users with unique roles, capabilities, and permissions with respect to implementing embodiments of the present invention. However, such accounts are listed for exemplary purposes only, and other embodiments of the present invention may include any number and/or any specific types of account as may be necessary to carry out the functions, features, and/or implementations of the present invention.

The civilian account is an account created by or for a civilian user who may implement various embodiments of the present invention in the user's civilian capacity. For example, civilian users may include non-emergency service personnel, discussed in more detail below, who use embodiments of the present invention to request and/or obtain emergency services.

The ES account is an account created by or for an emergency service user (ES user) who may implement various embodiments of the present invention in the ES user's official capacity as an ES user. For example, ES users may include emergency service personnel, such as police officers, fire department personnel, emergency medical technicians (EMTs), 9-1-1 operators, or other governmental employees/contractors who use embodiments of the present invention to provide or facilitate emergency services, as will be discussed in greater detail below.

The administrative account is a user account established by or for an administrative user (i.e., an “admin user”) of embodiments of the present invention. The admin user may make use of various embodiments of the present invention to manage the online resource, the database, and other components of embodiments of the present invention. In additional embodiments of the present invention, the admin user may establish and manage the civilian and ES accounts, such that the admin user can add, delete, modify, enable, or disable civilian and ES accounts. Furthermore, the admin user may establish and manage the roles, capabilities, and permissions of each of the different types of accounts and the users thereof.

Although certain details and descriptions provided below discuss certain functions, features, and/or implementations of the present invention being carried out by a particular type of user or user account, it is hereby understood that such descriptions are provided simply for illustrative purposes. Thus, for example, certain functions and features described below as being performed by an ES user may likewise be performed by a civilian user, and such is similarly applicable to other types of users and user accounts as may be required to carry out various such functions and features of embodiments of the present invention.

In addition, because embodiments of the present invention may provide access to personal and/or health-related information of certain users, users may be required to verify their identity and to confirm their status as a valid user in order to access various portions of embodiments of the present invention. The status of a civilian user may be verified by the user capturing a digital image, via their computing device 204, of their driver's license, birth certificate, social security card, passport, or other similar verification document, and uploading the digital image to the server device 204. The status of an ES user may be verified by capturing and uploading verification documents, such as a government ID, a government license, a badge number, or the like. All information provided (i.e., entered and/or uploaded) by users is generally received, via the network 206, and may be stored on the server device 202 or associated database. Upon providing such verification documents, embodiments of the present invention allow for such verification documents to be reviewed and verified, such as by one or more of the admin users. In further embodiments, users may be required to enter or will otherwise be provided with a username and password/passcode, which may be required for the user to login to the user's account and to access the electronic resource. Additional embodiments may provide for the user to select and provide answers to one or more security questions. For example, a security question may be “What is your mother's maiden name?”. The user may provide a security answer to the security question. If the user cannot remember the user's username or password/passcode, the user may be prompted to answer the one or more security questions so as to verify the user's identity.

Although certain embodiments of the present invention may require a user to establish an account, certain other embodiments may provide for certain features of the present invention to be utilized, such as viewing/browsing the electronic resource without requiring the establishment of a user account. For instance, a potential civilian user may access the online resource, such as an online website, and browse the functions and features available via embodiments of the present invention without first obtaining an account.

Embodiments of the present invention provide for users to obtain efficient and effective emergency services. As will be described below, such emergency services are broadly defined to include obtaining generally any type of emergency service, such as requesting emergency services, communicating with emergency service users, or otherwise obtaining information necessary to manage an emergency event. These, including other emergency services, are facilitated via embodiments of the present invention will be described in more detail below.

### Civilian Account

Before a civilian user can use certain functions and features of embodiments of the present invention, the civilian user will be required to create a user profile that can be associated with the user's civilian account. The user profile will include a plurality of types of information/data associated with the user, which can be used to facilitate the provision of emergency services to the user. The types of user profile information/data may include: personal information, health information, financial information, residential information, vehicle information, and insurance information. Such information may be entered and uploaded manually, via the GUI of the user's computing device 204. For example, embodiments of the present invention may provide for the civilian user to enter the information in “free-form” input fields (i.e., data entry areas) in response to prompts. As an illustrative example, if the civilian user is prompted to enter the user's birthdate, embodiments of the present invention may provide for the user to manually type in the user's birthdate (e.g., in the form Month/Day/Year) via a free-form input field generated and presented on the GUI. In other embodiments, the civilian user may be prompted to enter such information by selecting an appropriate selection from list of pre-selected responses, such as via “multiple-choice” responses, “scroll-down” responses, “slidable scale” responses, or the like. For example, the user may be presented, via the GUI, a set of scroll-down input fields that permits the civilian user to select the user's Month, Day, and Year of birth from a list of each possible month, days, and years. In still other embodiments, the user may obtain an image, via the camera of the user's computing device 204, of an item that includes various portions of the user's user profile information/data. Once obtained, embodiments of the present invention include image recognition algorithms capable of extracting textual data from the image. Thus, as will be described below, the user can simply obtain an image of an item that includes portions of the user's user profile information/data, and embodiments will automatically extract the necessary user profile information that is to be associated with the civilian user's user account.

The personal information portion of the user profile may include for example: full name (i.e., first name, middle name, surname), date of birth (DOB), social security number (SSN), location of birth (i.e., Country, State, City, etc.), marital status, height, weight, eye color, hair color, skin tone, body type, and blood type. In additional embodiments, the personal information may include: the total number of persons living in the user's residence (including children), how many children the user has, and the full name and age of each of the user's children. In further embodiments, the personal information may include: a name of the user's employer, a title of the user's position at the employer, an address of the user's employer, the user's work telephone number, the user's work email address, the user's supervisor name, the supervisor's telephone number, and the supervisor's email address. In still further embodiments, the personal information may include emergency contact information, such as: an emergency contact's name, relationship (e.g., spouse, parent, child, etc.), home address, phone number, email, social media account (e.g., Facebook, Twitter, LinkedIn, etc.).

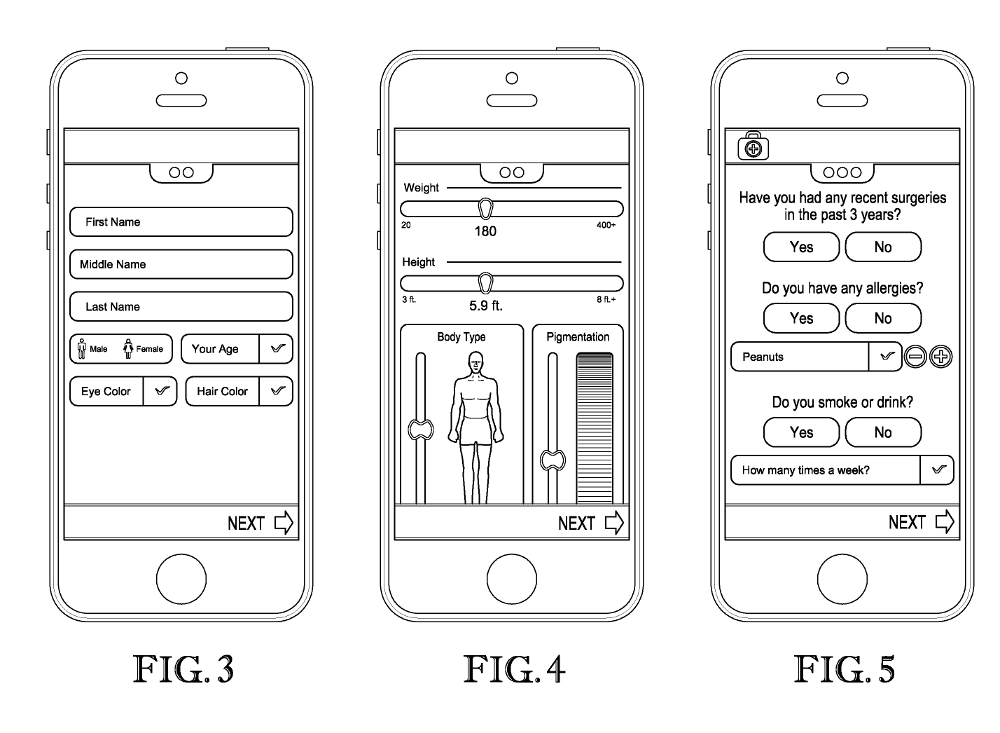

As previously described, such personal information may be entered by the user via free form input fields, or alternatively, via a selection of one or more preselected responses. In some embodiments, as illustrated in FIGS. 3-4, the preselected responses may be presented to the user, via the GUI, in graphical form so as to facilitate the user's ability to select an appropriate response. For example, for the case of skin tone, the civilian user may be presented with a plurality of skit tones (i.e., pigmentations, colors, etc.) from which to choose. Such plurality of skin tones may be presented simultaneously for selection, or in alternative embodiments, may be presented in a “scrollable” format such as through a virtual stylus wheel, a slidable bar, or the like. Similarly, for the case of body type, the civilian user may be presented with a plurality of virtual body styles outlined in a silhouette form. For example, such body styles may include the following weight types: obese, heavy, overweight, average, toned, and athletic. Similarly, such body styles may include the following height types: very short, short, average, tall, very tall. In some embodiment, such body styles may be selected from a “scrollable” format such as through a virtual stylus wheel, a slidable bar, or the like.

In some embodiments the personal information may be input by obtaining digital images of personal items via that civilian user's computing device 204. For example, the civilian user may obtain an image of the user's driver's license, birth certificate, social security card, passport, or the like. As previously described, embodiments of the present invention may obtain such personal information by scanning the digital images and extracting the personal information (e.g., name, DOB, address, etc.). In other embodiment, the digital images of the personal items may themselves be stored so as to represent the personal information. As such, digital images of the civilian user's driver's license, social security card, birth certificate, etc., may each be captured and stored in the memory elements of the civilian user's computing device 204 and/or in the server device 202. In still other embodiments, the civilian user may obtain a digital image of the civilian user, which may itself be operable to represent certain portions of the required personal information. For example, such a digital image of the user may be operable to represent the user's eye color, skin tone, hair color, height, weight, etc. in some embodiments, such personal information (eye color, skin tone, hair color, etc.) may be extracted from the digital image via image recognition software.

As illustrated in FIG. 5, the health information portion of the user profile may include for example: whether the user has any known allergies (if yes, the user may be able to choose from a list of possible allergens, e.g., foods, medicines, animals, etc.), whether the user has had a recent surgery (e.g., within 3, 5, or 10 years), whether the user smokes and/or drinks (if yes, the user may be able to choose from an amount of weekly smokes/drinks, e.g., 1, 5, 10, or more smokes or drinks each week), whether the user has any disabilities (e.g., blind, deaf, wheelchair disabled, etc.), whether the user is an organ donor, the user's primary physician (including name, address, phone number, email, etc.), and/or the user's primary dentist (including name, address, phone number, email, etc.).

The financial information portion of the user profile may include for example: the user's bank name, checking/savings account number, checking account routing number, debit card information (e.g., card type (VISA/MasterCard), primary account number (PAN), expiration date, card verification code (CVC), etc.), and/or credit card information.

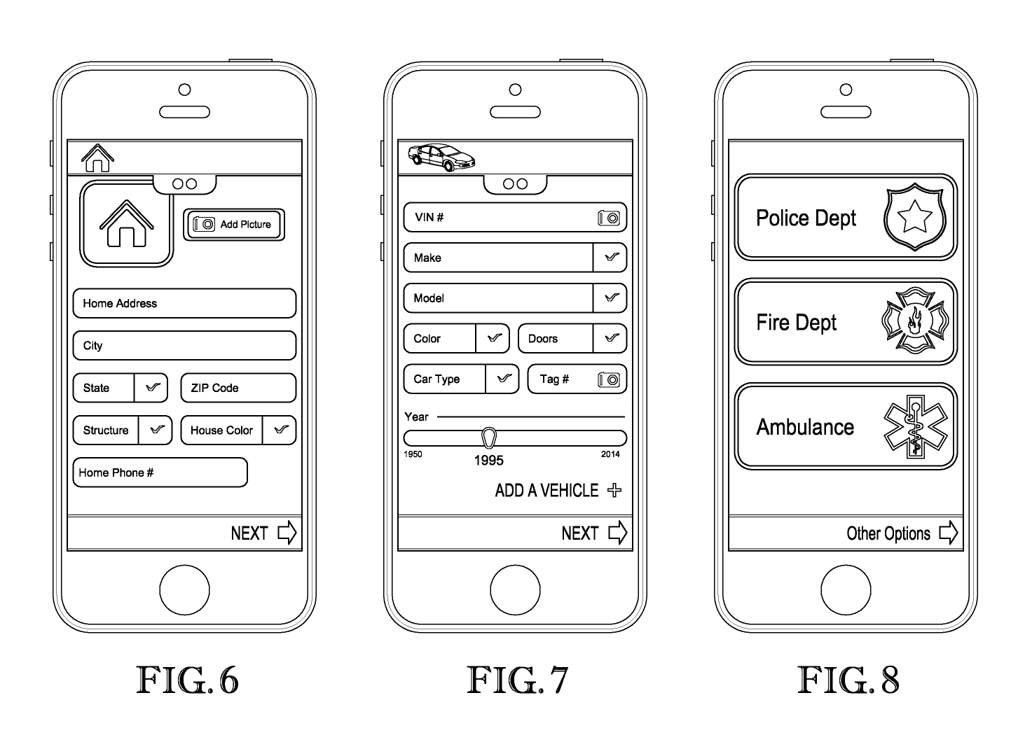

As illustrated in FIG. 6, the residential information portion of the user profile may include for example: the user's residential address, a color of the user's residence, a list of construction materials comprising the user's residence (e.g., wood, brick, metal, stucco, etc.). In certain embodiments, the user can obtain one or more digital images, via the computing device 204, of the interior and/or exterior of the user's residence and upload such digital images to the server device 202.

With reference to FIG. 7, the vehicle information portion of the user profile may include for example: a vehicle make, model, year, vehicle information number (VIN), license plate number, number of doors (e.g., 2 door or 4 door), general vehicle type (e.g., sedan, truck, minivan, SUV, motorcycle, etc.). In certain embodiments, the user can obtain one or more digital images, via the computing device 204, of the interior and/or exterior of the user's vehicle and upload such digital images to the server device 202. For example, the user may obtain images of the VIN, license plate number, or the general exterior of the vehicle.

The insurance information portion of the user profile may include for example: health insurance provider, health insurance policy number, names of individuals on the health insurance policy, contact information of the health insurance provider (e.g., account representative name, address, phone number, email, etc.). In other embodiments, the insurance information portion of the user profile may include for example: vehicle insurance provider, vehicle insurance policy number, and contact information of the vehicle insurance provider (e.g., account representative name, address, phone number, email, etc.). In additional embodiments, the insurance information portion of the user profile may include for example: residential (e.g., home/rental) insurance provider, residential insurance policy number, and contact information of the residential insurance provider (e.g., account representative name, address, phone number, email, etc.). In further embodiments, the insurance information portion of the user profile may include for example: life insurance provider, life insurance policy number, life insurance beneficiaries, and contact information of the life insurance provider (e.g., account representative name, address, phone number, email, etc.). ES Account

Turning to the ES user, before such an ES user can implement certain functions and features of embodiments of the present invention, the ES user will be required to create a user profile that can be associated with the ES user's ES account. The user profile will include a plurality of pieces of identification information associated with the ES user, which can be used to facilitate the provision of emergency services to civilian users. The identification information may include: an ES user's name, a type of ES user (e.g., police officer, fire department personnel, EMT, etc.), a name of governmental unit/department to which the ES user is associated, an address of the governmental unit/department, a phone number of the governmental unit/department, an email address of the governmental unit/department, or the like. Such information may be provided (i.e., entered and uploaded) manually by the ES user, via the GUI of the ES user's computing device 204. In certain embodiments, the identification information may be obtained by capturing and uploading identification documents, such as a government ID, a government license, a badge number, or the like. Image recognition software may, thus, be capable of extracting the required identification information from the identification documents, as was previously described with the civilian user.

In other embodiments, the ES user's governmental unit/department may have access to embodiments of the present invention, such that the unit/department may generate an ES user account for each ES user within the unit/department. As such, the unit/department may be responsible for generating the user profile identification information for each ES user. As an illustrative example, if a city police department includes a force of five police officers, the police department may create five ES accounts and associate each of its five police officers with an ES account. When establishing the ES accounts, the police department may associate the identification information for each police officer with their respective ES account.

### Operation

### Civilian User Applications

In operation, embodiments of the present invention are capable of providing various functions and feature to be available to each of the civilian and ES users. Broadly, embodiments of the present invention facilitate the provision of emergency services to civilian users. In more detail, when a civilian user is undergoing a particular type of emergency event, such as a criminal emergency, a fire emergency, a medical emergency, or the like, the civilian user can access the electronic resource of embodiments of the present invention, via the user's computing device 204, to obtain emergency services. For example, and with reference to FIG. 8, upon the civilian user accessing the electronic resource, the civilian user will be presented with options of whether the civilian user is in need of emergency service for a particular type of emergency event, such as a criminal-type emergency event (i.e., “Police Dept.”), a fire-type emergency event (e.g., “Fire Dept.”), or a medical-type emergency event (i.e., “Ambulance”). Embodiments of the present invention are operable, then, to determine which type of ES user to which to generate a request to provide emergency services for the civilian user. Such a determination is based, in part, on the user profiles of those ES users participating in embodiments of the present invention. As previously described, the user profile of each ES user indicates the type of emergency services personnel (e.g., police-type, fire department-type, EMT-type). As such, for a criminal-type emergency event, embodiments will generate a request that a police-type ES user provide emergency services. For a fire-type emergency event, embodiments of the present invention will generate a request that a fire department-type ES user provide emergency services. For a medical-type emergency event, embodiments of the present invention will generate a request that an EMT-type ES user provide emergency services.

Upon requesting that the appropriate type of ES user provide emergency services, a communications link may be generated, via the communications network 206, between the civilian user, via the civilian user's computing device 204, and an appropriate ES user, via the ES user's computing device 204. In certain embodiments, the communication link may be via text message (e.g., SMS, MMS, email, etc.), voice communication, or video communication (e.g., Facetime™, Skype™, Viber™, Tango™, Zoom™, or the like). For example, the text, voice, or video communication link may be established via the communications network 206, which may include standard cellular networks (e.g., 3G or LTE) or VoIP, such as via WiFi. As such, upon requesting emergency services from the appropriate ES user, the civilian user can immediately begin communicating directly with the ES user.

In some embodiments, the civilian user may automatically request to obtain the services of a ES user via an automated alert originating from an electronic device associated with the civilian user. For example, the electronic device may be selected from one or more of the following: a pacemaker, a heart-rate monitor, a blood-pressure monitor, a home security system, or a home fire detection system. Such electronic devices may be electronically connected to the computing device 204 of the civilian user, such as through Bluetooth™, WiFi, or the like. Alternatively, the electronically devices may be directly connected to the server devices 202 via the communications network 206. As such, when the electronic device detects a particular event, embodiments may provide for the electronic device (independently or via the computing device 204) to automatically request to obtain emergency services from a particular type of ES user. For example, if the civilian user has a heart condition that requires the civilian user to wear a pacemaker, the pacemaker may be an electronic device that is operable to automatically request (independently or via the computing device 204) emergency services. For instance, if the civilian user's heart rate/rhythm reaches a dangerous level, the pacemaker may generate an automate alert (independently or via the computing device 204) to request emergency services. Embodiments of the present invention will recognize that the alert has been generated from a pacemaker and thus request the emergency services from an EMT-type ES user. Similarly, in the case of a home security system being an electronic device, should the security system detect an intruder in the civilian user's home, the security system may generate an automate alert (independently or via the computing device 204) to request emergency services. Embodiments of the present invention will recognize that the alert has been generated from a security system and thus request the emergency services from a police-type ES user. As a further example, in the case of a home fire detection system being an electronic device, should the home fire detection system detect a fire in the civilian user's home, the fire detection system may generate an automate alert (independently or via the computing device 204) to request emergency services. Embodiments of the present invention will recognize that the alert has been generated from a home fire detection system and, thus, request the emergency services from a fire department-type ES user. Regardless of the type of emergency service requested, once the emergency service has been requested from a particular ES user, embodiments of the present invention will provide for the ES user to establish a communications link with the civilian user via the civilian user's computing device 204.

In further embodiments, the civilian user may not be immediately placed into a communications link with an intended ES user (e.g., police, fire, EMT). Instead, the civilian user may first be placed in communication with an intermediary ES user, such as an emergency services operator (i.e., 9-1-1 operator), who facilitates the eventual establishment of a communications link between the civilian user and the intended ES user.

In still further embodiments, before a communications link is established, the civilian user may be prompted to respond to whether the civilian user is capable of communicating audibly. For example, in certain criminal and/or medical emergency events, the civilian user may be unwilling or unable to communicate audibly. If the civilian user responds by indicating that the civilian user is unable to communicate audibly, only a text message-type communications link may be established, such that voice and/or video communication is not permitted. Furthermore, certain embodiments may provide for a voice-to-text capability, such as used in Dragon Speech™, Apple Siri™, or Google Voice™. For example, the ES user may be capable of speaking, audibly, into the speaker of the ES user's communication device 204, and embodiments of the present invention will convert the ES user's voice communication into one or more text messages (i.e., voice-to-text), which can be sent to the civilian user. As such, the ES user is capable of communicating with the civilian user, without the civilian user's computing device 204 emitting audible noises. In some embodiments, voice communications are capable of being continuously converted to text messages and periodically (e.g., every 5, 10, 30 seconds) sent to the intended user. Such embodiments may also be used to convert audio portions of video communications into text messages (i.e., video-to-voice-to-text). In additional embodiments, text message communications may, alternatively, be converted to voice communications (i.e., text-to-voice). As such, text messages submitted by a civilian user to an ES user may be converted via the ES user's computing device into a voice communication.

In even further embodiments, the communications device 204 is operable to capture digital images and/or videos, and embodiments of the present invention are operable to detect patterns in the images and/or videos and convert such patterns into text. For example, such an embodiment may be used to capture sign-language communications performed via hand positions and/or hand motions of a user and convert such sign-language communications into textual communications, such as is performed using the Kinect Sign Language Translator™. In some embodiments, such functionality may be facilitated via a separate program connected to embodiments of the present invention via an API. Nevertheless, civilian users who may be hearing impaired or who are otherwise unable to speak, may communicate with ES users by performing sign-language in view of the camera of the computing device 204. Via image recognition algorithms, embodiments of the present invention are operable to capture such sign-language communications and convert such sign-language communications into one or more text messages that are understandable by ES users. In other embodiments, sign-language communications and/or text message communications may be converted into a video communication (e.g., text-to-voice), such that ES users can hear a voice translation of the sign-language and/or text message communications. In still further embodiments, the voice and/or text communications may be converted/translated into graphic illustrations of sign-language that may be presented on the GUI of the civilian user's computing device 204. For example, the GUI may present a graphic illustration of a hand or a person's body performing sign language. As such, a hearing impaired civilian user can observe the sign-language communications that have been translated from the ES user's original voice and/or text communications.

Embodiments of the present invention further include the capability to translate communications from a plurality of first languages into a plurality of second languages. For example, a civilian user that speaks/writes only in Spanish may communicate in Spanish, via the civilian user's computing device 204, and embodiments of the present invention are operable to convert such Spanish communications to English communications for presentation to the ES user. In the case of text messages, embodiments of the present invention may include (or connect to via API) a translation function, such as is implemented in Google Translate™, which is capable of translating a text message entered by a first user in a first language into a text-message presented to a second user in a second language. In additional embodiments, the voice-to-text feature may be combined with the translation feature, such that a first user can provide a voice communication in a first language, with such voice communication being converted to a text message in the first language. Thereafter, embodiments may translate the text message in the first language to a text message in a second language, with such second language being understood by a second user. In still further embodiments, the text message in the second language may further converted into voice communication (i.e., text-to-voice), such that the second users is able to hear an audio translation of the original voice communications. As such, users (including both civilian and ES users) that speak different languages are able to communicate such that the users can obtain and provide emergency services via embodiments of the present invention.

In some embodiments, before, during, or after the communications link with the ES user has been established, the civilian user will be prompted to provide certain pieces of information, in the form of event descriptions, which describe the emergency event that is occurring. Such event descriptions will be provided to the ES user via the ES user's computing device. In some embodiments, the civilian user will be presented, via the GUI, as a free-form input field into which the civilian user can input event descriptions in in the form of textual descriptions of the emergency event that is occurring. Such free-form textual descriptions may include any type of relevant information that the civilian user thinks is applicable to the emergency event and/or may be beneficial to the ES user that is to be providing emergency services. For example, in a medical-type emergency event, the civilian user may indicate that the civilian user is “having shortness of breath,” “experiencing pain in the left arm and in the chest,” and/or “is feeling lightheaded.” As such, the ES user may use such event descriptions as indications that the civilian user is experiencing a heart attack. Thus, the ES user can be better prepared to offer emergency services to the civilian user. As an additional example, a civilian user that is experiencing an emergency situation, in the form of being lost or stranded, may provide various types of event descriptions, which may assist an ES user in finding the civilian user and providing appropriate emergency services to the civilian user. For example, the civilian user may provide the following descriptions as part of the free-form input field: last known position (e.g., latitude/longitude/altitude), wind speed, current strength (in the case of being lost at sea), level of light, temperature, distance from land/shore, depth (in the case of being lost under the sea), and any other descriptions of visible items (e.g., boats, structures, aircraft, star field orientation, etc.).

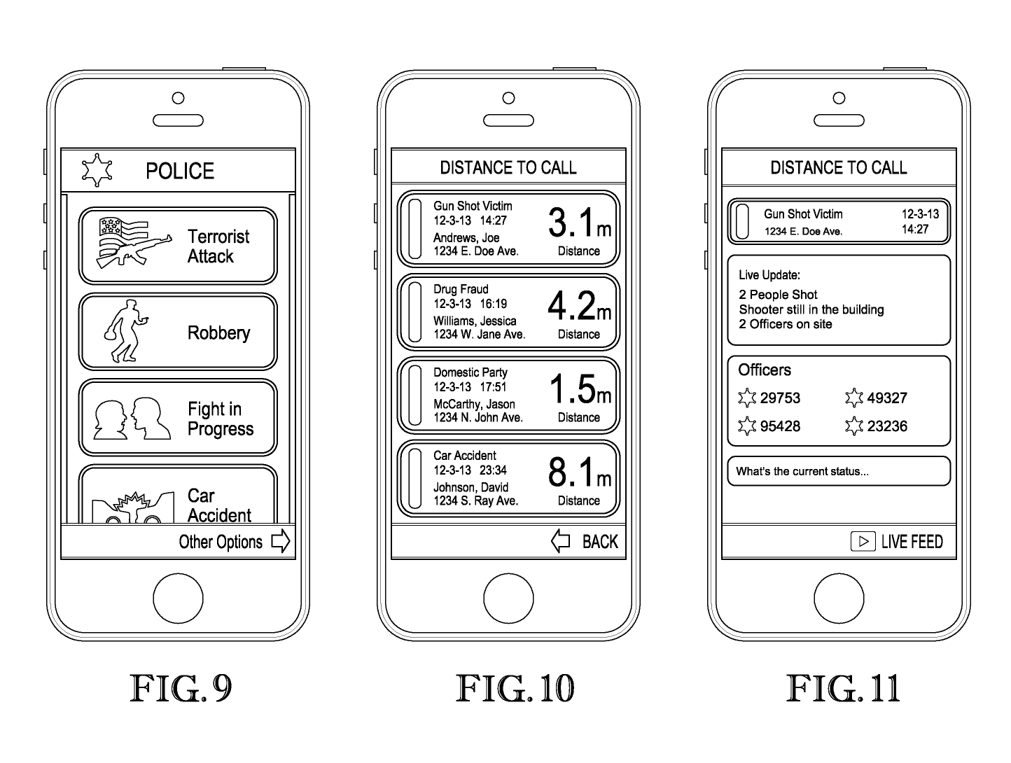

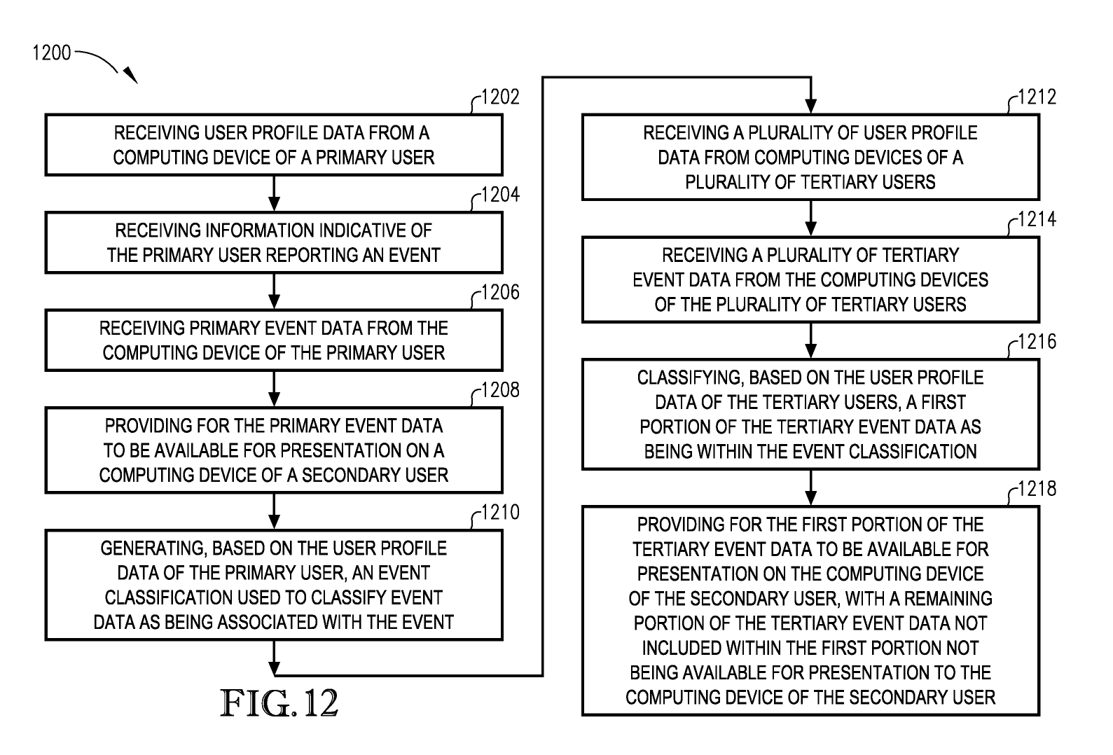

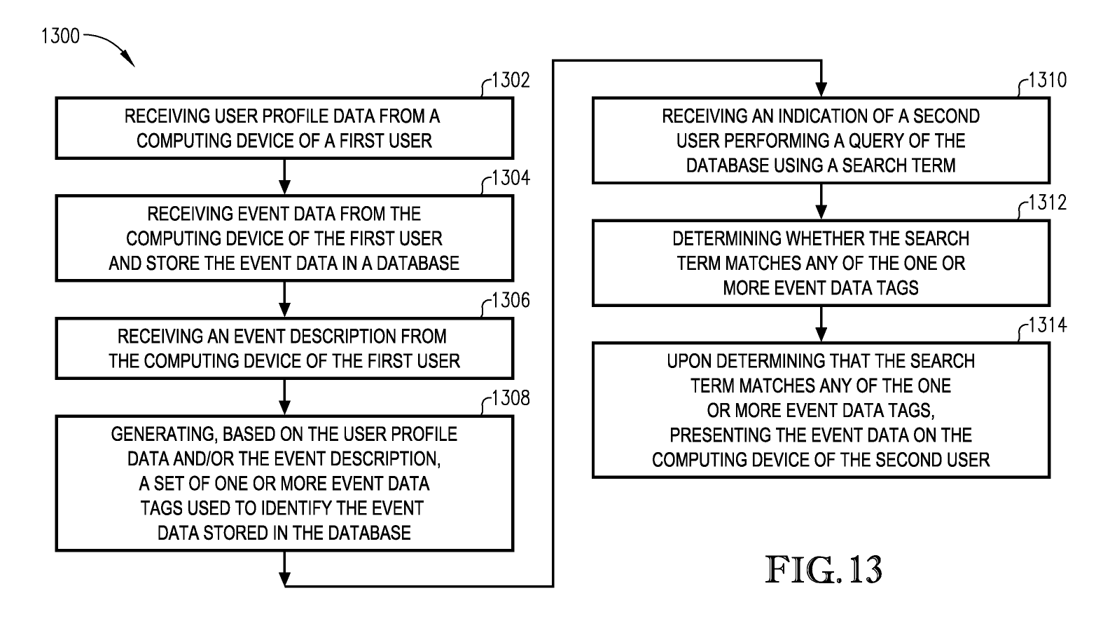

In other embodiments, the civilian user will be presented with a plurality of pre-selected questions to which the civilian user provides answers. In such embodiments, the civilian user's answers may comprise the event descriptions. In certain embodiments, the pre-selected questions are presented based on the type of emergency event identified by the civilian user. For example, and with reference to FIG. 9, if the civilian user is undergoing a criminal-type emergency, the civilian user may be asked “Is this a terrorist attack?”, “Is this a robbery?”, “Is there an altercation in progress?”, “Has there been a car accident?”, etc. The civilian user may simply provide “yes/no” type answers to the pre-selected questions. Furthermore, any additional pre-selected questions may be selected based on the answers provided by civilian users to earlier questions. For example, if the civilian user indicated that the criminal-type emergency was a “robbery,” additional pre-selected questions may be “What was the thief wearing?,” “did the thief use a weapon?,” “what did the thief steal?”, and/or “which direction di the thief go?”, etc. As such, the ES user of which the emergency services are requested will be provided with the answers (i.e., the event descriptions), such that the ES user can understand the emergency event in more detail and can be better prepared when rendering emergency services.

In addition to establishing a communications link (e.g., text, voice, video) between the civilian user and the ES user, embodiments of the present invention provide for various portions of the civilian user's user profile to be provided to the ES user. The user profile includes information that can be used to inform the ES user of civilian user-related information that may be beneficial for the ES user to efficiently and effectively provide emergency services to the civilian user. As an illustrative example, in a medical-type emergency event in which the civilian user reports a medical emergency, the ES user (e.g., an EMT) will be provided with the civilian user's listed allergies. As such, the ES user can be prepared to provide emergency services by ensuring to carry appropriate medicines that can treat the civilian user without causing an allergic reaction. As an additional illustrative example, in a fire-type emergency event in which the civilian user reports a fire at the user's residence, the ES user (e.g., a fireman) will be provided with the building material of which the civilian user's residence is constructed (e.g., wood or brick). As such, the ES user can be prepared by ensuring to carry appropriate fire-extinguishing tools, which may be necessary to fight a fire in a residence constructed of such building material. As a further illustrative example, in a criminal-type emergency event in which the civilian user reports a “break-in” at the user's residence, the ES user (e.g., a police officer) will be provided with the number of persons living in the civilian user's residence (e.g., spouse and children). As such, when the ES user arrives at the civilian user's residence, the ES user will understand that the ES user should expect to find the civilian user and the additional persons living in the civilian user's residence.

Furthermore, embodiments of the present invention provide for the civilian user to capture various types of event data, with such event data operable to account and/or chronicle the emergency event that is taking place. In more detail, the civilian user can capture text, video, and image data, via the civilian user's computing device 204, and stream the video/image data to the ES user's computing device 204 via the communications network 206. For example, in a fire-type emergency event, in which the civilian user reports a fire at the user's residence, the civilian user can stream a live video of the fire to the ES user (e.g., fireman). As an additional example, in a criminal-type emergency event in which the civilian user reports an “altercation” between third-parties, the civilian user can stream live video of the altercation to the ES user (e.g., policeman). In addition to providing for live streaming of event data, embodiments of the present invention provide for event data (video and/or image data) to be stored in the server device 202 and/or computing devices 204 for later use, such as for review and analysis.

In certain embodiments, before a communication link is established between the civilian user and the ES user and/or before an ES user will provide emergency services to the civilian user, the ES user may first be provided with a request to provide emergency services, via the ES user's computing device 204. As such, the communications link will not be established and/or the ES user will not provide emergency services until such time as the ES user accepts the request to establish the communications link and/or to provide emergency services. Furthermore, at any given time, the ES user may be provided with a plurality of requests to establish communications links and/or to provide emergency services. As provided in FIG. 10, the ES user may be presented with a request to establish a plurality of communications links and/or to provide emergency services to a plurality of civilian users. In such embodiments, the ES user may select which civilian user to establish a communications link with and/or to provide emergency services to bases on various factors, including: elapsed time of request, distance/travel time between the ES user and the civilian user (as determined by geo-location, as will be discussed in more detail below), and the type of emergency event. Such embodiments of the present invention, with respect to the ES user's selection of which emergency events to respond, will be discussed in more detail below.

As such, embodiments of the present invention are operable for generating a communication link between a civilian user and one or more types of ES users (e.g., police, fire, EMT, etc.). As previously provided and with reference to FIG. 1, embodiments include the method 100 with the initial Step 102 of receiving user profile data from a plurality of ES users, with the user profile data indicating whether each ES user in the plurality is an ES user of a first type or a second type. The next Step 104 includes providing a set of computer-executable instructions that, when executed by the civilian user's computing device, generates a user interface displayable on an electronic display of the computing device. Next Step 106 includes presenting, via the user interface of the civilian user's computing device, a solicitation for the civilian user to select an emergency event type. In Step 108, embodiments provide for receiving information indicative of the emergency event type. Thereafter, in Step 110, based on a comparison between the emergency event type and the user profile data of the ES users, embodiments send a notification to an ES user of either the first type or the second type, wherein the ES user to which the notification is sent is a first ES user. Step 112 includes providing a set of computer-executable instructions that, when executed by a computing device of the first ES user, generates a user interface displayable on an electronic display coupled to the first ES user's computing device. Next Step 114 includes presenting, via the user interface of the first ES user's computing device, a solicitation for the first ES user to communicate with the civilian user. In final Step 116, upon receiving information indicative of an assent to communicate with the civilian user, a communication link is opened between the first ES user and the civilian user, via the respective computing devices of the first ES user and the civilian user.

Embodiments of the present invention are additionally operable to obtain and track a geo-location of each civilian user via the civilian user's computing device 204. In certain embodiments, the user's geo-location is included as part of the user's user profile. Furthermore, embodiments of the present invention provide for the user's geo-location to be updated in near real-time, such that the user's user profile is continuously updated to include the user's current and/or last known geo-location.

In some embodiments, the geo-location is determined via GPS receiver included in the civilian users' computing device 204. In other embodiments, the geo-location will be determined via cellular networks from the communications network 206. For example, the geo-location may be determined via network-based cellular triangulation, in which case such geo-location information may be received from cellular service providers. However, in other embodiments, the geo-location may be directly obtained, via the civilian user's computing device 204, through the computer program of embodiments of the present invention. For example, embodiments of the present invention may provide for the processing elements of the computing device 204 (under direction from the computer program of embodiments of the present invention) to determine a geo-location based on signal strengths of nearby cellular towers or WiFi hotspots. In particular, a geo-location of the civilian user can be determined by comparing the signal strengths of the cellular or WiFi signals from a plurality of cellular towers or WiFi hotspots, respectively. The accuracy of the civilian user's geo-location is improved with the increased number cellular towers or WiFi hotspots within range of the civilian user's computing device 204. As such, when determining a geo-location, it is preferred if there are at least three cellular tower or WiFi hotspots within range of the civilian user's computing device 204 at any given time.

A benefit of determining geo-location based on WiFi hotspots is that the geo-location can be determined in indoor locations (e.g., buildings, underground, etc.) or crowded venues (e.g., concerts, sporting events, etc.). In such locations/venues, cellular networks and/or GPS may be compromised or unavailable. In certain instances, embodiments of the present invention will provide for a geo-location determination based on a hybridized analysis of each of cellular, GPS, and WiFi signals, such as may be performed by Google Latitude™, AlterGeo™, and UonMap™. In even further embodiments, geo-location may be determined via Bluetooth™. Such a determination may be made by analyzing Bluetooth™ signal strengths of nearby Bluetooth™ capable devices, such as other civilian and/or ES user's computing devices 204. Regardless of the method of determining the civilian user's geo-location, such a geo-location may be constantly updated to the server device 204. Additionally, past geo-locations of the civilian user may be stored in the server device 204, such that other users (e.g., ES users) can review the previous locations and routes that that were taken by the civilian user.

As mentioned above, because a geo-location can be determined based on non-cellular/GPS communications networks, including WiFi and Bluetooth™, a precise geo-location of a civilian user can be determined within buildings, sports arenas, or the like. For example, if a civilian user is undergoing a medical emergency event while attending a sporting event at a sporting stadium, embodiments of the present invention are operable to determine the civilian user's precise position within the stadium, such as by seating section, row number, seat number, or the like. In more detail, if the stadium includes a plurality of WiFi hotspots, embodiments of the present invention can triangulate the civilian user's geo-location by analyzing and comparing a signal strength of the civilian user's computing device 204 to each of the plurality of WiFi hotspots. In certain embodiments, the server device 202 will store a plurality of engineering schematic and/or graphic illustrations of buildings and stadiums, which further facilitate determining the precise geo-location of users within the building or stadium. Remaining with the above example of a sports stadium, embodiments of the present invention may overlay the schematic of the sports stadium with the civilian user's geo-location to determine the precise area of the stadium (e.g., section, row, seat) in which the civilian user is located. As such, embodiments of the present invention can provide an ES user with specific directions necessary to travel from the ES user's geo-location to the civilian user's geo-location in the most efficient amount of time. Similarly, a civilian user's precise position within a building may likewise be determined. After such a geo-location has been determined, the geo-location may be overlaid on a graphic illustration of the building and presented, via the GUI of the ES user's computing device 204, such that the ES user can visually determine the precise geo-location of the civilian user.

The use of geo-location functionality facilitates various other specific features of embodiments of the present invention, such as may be implemented when the civilian user is in communication with the ES user. For example, once the communications link has been established between the civilian user and the ES user, an estimated arrival time of the ES user to the civilian user's location can be determined and presented. In more detail, the geo-location of the civilian user can be compared with the geo-location of the ES user to determine an estimated travel and/or arrival time of the ES user to the civilian user's geo-location. In some embodiments, the travel time will be based on a straight, line-of-sight distance between the civilian user and the ES user. In other embodiments, the travel time will incorporate route information, such as a drive time, on roads and highways, between the ES user and civilian user. In such embodiments, the drive time will incorporate the distance and speed limit of the roads and highways included in the route. Such distance and speed limit information may be obtained and stored within the server device 202, or alternatively, such distance and speed limit information may be obtained by accessing one or more open source resources, such as Google Maps™ or Yahoo Maps™, via an API.

In even further embodiments, traffic information may be obtained and incorporated in the route information, so as to adjust the travel time as needed. In some embodiments, the traffic information may be obtained from open source resources, such as Google Maps™ or Yahoo Maps™. In such instances, embodiments of the present invention may be operable to link with such open source resources, via one or more APIs. Alternatively, embodiments of the present invention may uniquely determine traffic information by analyzing the geo-location of each user of embodiments of the present invention. For example, in situations in which geo-locations of a plurality of civilian users indicate that such civilian users are stationary or moving very slowly on a particular section of highway, embodiments of the present invention may determine that the particular section of highway is congested with traffic. As such, any routes extending through such section of highway will have their travel time increased. In instances in which the indicated traffic congestion is overly high, embodiments of the present invention may propose an alternate route, and thus, re-calculate the travel time/response time appropriately.

Additional embodiments may use geo-location of a civilian undergoing an emergency event to determine a safe egress route from the emergency event. For example, if the civilian user is undergoing an emergency event (e.g., a terrorist attack) at a particular intersection of a city's streets, embodiments of the present invention may determine and present an egress route from the particular intersection via one or more roads or highways. Such an egress route may be presented graphically, via the GUI of the civilian user's computing device 204, by overlaying a graphic depiction of the egress route over a graphic map of the city and/or the city streets. Alternatively, the egress route may be presented textually, in the form of step-by-step instructions.

Furthermore, if the civilian user is undergoing an emergency event (e.g., a fire) inside a building, embodiments of the present invention may determine and present an egress route from the civilian user's geo-location within the building to a safe location outside of the building. As previously provided, such an egress route may be presented graphically, via the GUI of the civilian user's computing device 204, by overlaying a graphic depiction of the egress route over a graphic illustration (or schematic) of the building. In certain embodiments, such a graphic illustration may be in the form of a 3D rendering of the building. Such a depiction may be enhanced by overlaying the egress route over the graphic schematic of the building stored in the server devices 202. Because embodiments of the present invention provide for the civilian user's geo-location to be continuously updated and presented graphically to the ES user, the ES user can monitor whether the civilian user is following the egress route or if the civilian user has deviated from the egress route. In instance in which the civilian user has deviated from the initially-provided egress route, embodiments of the present invention my re-calculate a new egress route based on the civilian user's most recent geo-location.

In even further embodiments, ES users can generate and provide unique egress routes to civilian users. For example, the geo-location of a civilian user may be displayed on a graphic map, via the GUI of the ES user's computing device 204. Using the input controls (e.g., touchscreen, mouse, etc.) of the ES user's computing device 204, the ES user can draw a unique egress route on the graphic map, with such egress route being graphically depicted in the form of a colored line(s), breadcrumbs, or the like. As such, the graphic map, along with the egress route depiction, can be transmitted to the civilian user's computing device 204 for display, such that the civilian user can observe the unique egress route and take the route to safety.

### ES User Applications

Returning to the previously-described illustration of embodiments of the present invention in which a civilian user has requested to receive emergency services from an ES user, which includes establishing a communications link with an ES user, once the civilian user has made such a request, embodiments of the present invention will generate an event classification for the event. The event classification is used to identify and group together all information and data relevant and/or related to the emergency event. For example, all of the user profile data, all of the event descriptions, and all of the event data provided by the civilian user undergoing the emergency event will be grouped into an event classification that has been generated via embodiments of the present invention. As will be described in more detail below, embodiments of the present invention generate the event classification so as facilitate the association of a plurality of data with a particular event.

Returning to FIG. 10, an ES user will be presented, via the GUI of the ES user's computing device, with a listing of all emergency events that have been reported by civilian users, with such listing further representative of civilian users having requested emergency services (including a request to establishing a communications link) from the ES user. As illustrated in FIG. 10, each emergency event listed and presented to the ES user will be associated with a particular emergency event. As previously mentioned, the types of emergency events that are presented to ES users are determined, in part, on a comparison between the type of emergency event and the type of ES users. For example, embodiments of the present invention may provide for police officer-type ES users to be presented only with a listing of criminal-type emergency events, fire department-type ES users may be presented only with at listing fire-type emergency events, and EMT-type ES user will be presented only with a listing of medical-type emergency events.

Furthermore, those ES users that are presented with a particular listing of emergency events may be determined based on other factors, such as distance/travel time from the ES users to the particular civilian and/or to the emergency event. For example, if a civilian user is undergoing a criminal emergency, once the civilian user selects to obtain emergency services from an ES user, embodiments of the present invention may present the civilian user's request for emergency services to all police-type ES user's having a geo-location within a 1 mile radius from the geo-location of the civilian user.

Regardless of how a particular ES user is selected to receive the listing of emergency events, as illustrated in FIG. 10, once the ES use has been presented with the listing, the ES user may select which emergency event to respond and which civilian user with which to establish a communications link. Such a decision may be based on various factors, such as: elapsed time of the civilian user's request, distance/travel time between the ES user and the civilian user (as determined by geo-location), and the type of emergency event. For example, the ES user may choose (or may be required by departmental policies) to first respond to and/or and establish a communications link with the civilian user who has a geo-location that is the closest to a geo-location of the ES user. Alternatively, the ES user may choose (or may be required by departmental policies) to first respond to and/or and establish a communications link with the civilian user who has a geo-location with the shortest travel time from the geo-location of the ES user. Embodiments provide for the ES user's GUI to display the distance and/or travel time to each of the listed emergency events/civilian users. Alternatively, the ES user may choose (or may be required by departmental policies) to first respond to and/or establish a communications link with the civilian user who first requested the emergency services. Embodiments provide for the GUI to display the time of which each request was made, such as in the form date/hour/minute.

In further alternatives, the ES user may choose (or may be required by departmental policies) to first respond and/or establish a communications link with the civilian user who is undergoing the most significant emergency event. The determination as to which emergency event is more or less significant may be based, in part, on the event descriptions provided by the civilian user to the pre-selected questions presented to the civilian user, as previously described (see FIG. 9). Such answers may be displayed via the GUI of the ES user's computing device. For example, if the civilian user indicates that the emergency event is a terrorist attack, such an emergency event may be more significant, and thus have a higher priority, than an emergency event indicated to be a vehicle accident. In certain embodiments, the significance level of the emergency may be presented to the ES user in the form of a color-code on the ES user's computing device 204. For example, a “red” color-code may represent a most significant situation (e.g., a shooting situation with injuries). A “yellow” color-code may represent an intermediate significant situation (e.g., an altercation with minimal injuries). And a “green” color-code may represent a minimal significant situation (e.g., a non-violent drug situation)

Once an ES user has selected an emergency event to which to respond and/or a civilian user with which to provide emergency services, a communications link may be established between the ES user and the selective civilian user. However, in certain embodiments, before the communications link is established, the ES user will be presented with a summary screen, such as illustrated in FIG. 11, which provides more detail as to the selected emergency event. For example, the summary screen may include various portions of the civilian user's user profile, a geo-location of the civilian user, any event descriptions provided by the civilian user, or any event data previously uploaded by the civilian user. In other embodiments, if other ES users have already responded to the emergency event, any event data or event descriptions provided by such ES users may also be provided via the summary screen. In such embodiments, the summary screen may also list each of the other ES users that have responded to the emergency event. The other ES users that have responded to the emergency event may include those other ES users that accepted to respond to the emergency event via embodiments of the present invention. Alternatively, the other ES user presented via the summary screen list may be determined by including those ES users within a particular distance from the geo-location of the emergency event and/or civilian user. As with the civilian user, the geo-location of ES user may also be determined by cellular, GPS, WiFi, or Bluetooth™ networks, or combinations thereof. For example, in certain embodiments, the summary screen may present a list of other ES users that have a geo-location within 1 block, ¼ mile, ½ mile, 1 mile, 2 miles, or more than 2 miles from the geo-location of the emergency event and/or the civilian user.

Upon selecting an emergency event to which to respond and upon establishing a communications link with a civilian user, the ES user is able to review user profile data, event descriptions, and event data provided (i.e., uploaded or live-streamed) by the civilian user who is undergoing the emergency event, as previously described. For example, if a civilian user is undergoing fire-type emergency event, the ES user with which a communications link has been established is able to review event data (e.g., images/videos) of the fire that was uploaded and/or streamed by the civilian user. In the case of event data that is being streamed, the ES user is capable of viewing the event data live via the ES user's computing device 204. In the case of event data that has been uploaded/saved on the server device 202, the ES user is capable of viewing, rewinding, fast-forwarding, playing in slow motion, zooming in/out, and otherwise analyzing the event data.

In addition to the civilian user that undergoing the emergency event being able to provide event data (e.g., image and video data related to the emergency event), embodiments of the present invention provide for other civilian users and/or other ES users to obtain and upload their own event data. In more detail, it is understood that a plurality of civilian users and ES users may be using embodiments of the present invention simultaneously. As such, in addition to the civilian user undergoing the emergency event having the ability to upload event data, other civilian users and/or ES users may be able to obtain event data of the emergency event may be able to stream and/or upload such event data via embodiments of the present invention. Embodiments of the present invention provide for the ES user that responds to the emergency event and that established a communications link with the civilian user to review the event data provided by such other civilian users and other ES users. In particular, such event data may be presented/viewed on the ES user's computing device 204, similar to how the event data provided by the civilian user undergoing the emergency event is presented/viewed.

To determine whether event data obtained by other civilian users and/or other ES users may be related to the emergency event being undergone by a particular civilian user, embodiments of the present invention analyze user profiles, event data, and event descriptions of the other civilian users and/or other ES users to determine whether such user profiles, event data, and event descriptions are classified within the event classification of the emergency event being undergone by the particular civilian user.

In more detail, the event classification will be determined based on portions of the user profile or on portions of the event description provided by the civilian user who is undergoing the emergency event. For instance, in some embodiments, the event classification will be based on the geo-location of the civilian user, the home address of the civilian user, the area code of a telephone number associated with the civilian user, or a date/time stamp of when the civilian user requested emergency services from an ES user. As an illustrative example, the event classification may be based on the geo-location of a particular civilian user undergoing an emergency event. Specifically, the event classification may be generated based on a geographic area having a boundary within a given range from the geo-location of the civilian user. For example, in some embodiments, the given range may be about 1 block, ¼ mile, ½ mile, 1 mile, 2 miles, or more than 2 miles from the geo-location of the civilian user. As such, event data that is provided by other civilian users that have a geo-location within the given range may be classified within the event classification, such that the event data may be presented to the ES user responding to the emergency event of the particular civilian user. Event data that is provided by other civilian users that do not have a geo-location within the given range may not be classified within the event classification, such that the event data may not be presented to the ES user responding to the emergency event. Nonetheless, it is understood that all event data, whether or not classified as part of any emergency event classification, will be saved in the database of the server device 202 for later use, as may be required. In other embodiments, the event classification may be generated based on a geographic area having a boundary within a given range from the home address of the particular civilian user, from an area identified by the area code of the phone number of the particular user, or other similar geographically defined areas associated with the particular civilian user.

In other embodiments, the event classification may be generated based on a timing (i.e., date/time stamp) of the emergency event. For example, the event classification may be defined as encompassing a given timeframe before the emergency event and extending through a given timeframe after the emergency event. As an illustrative example, the given timeframe may be 1 minute, 5 minutes, 15 minutes, 30 minutes, 1 hour, or more. As such, event data that is provided by other civilian users within the given timeframe before and after the date/time stamp associated with emergency event will be classified within the event classification, such that the event data may be presented to the ES user responding to the emergency event of the particular civilian user. On the other hand, event data that is provided by other civilian users not within the given timeframe of the date/time stamp may not be classified within the event classification, such that the event data may not be presented to the ES user responding to the emergency event.

Embodiments provide for the event classification to be based on various other portions of the user profile data, the event data, and/or the event description as may be necessary or preferred. Furthermore, event data provided by other ES users may similarly be classified in the event classification, such that the event data may be presented to the ES user that is responding to the emergency event. In still other embodiments, event data that is captured and provided by non-user assets may be obtained and classified as being either within or outside of the event classification. Such non-user assets may be permanently-positioned assets, such as security systems and/or image/video cameras. For instance, such permanent assets may comprise video cameras associated with security systems for buildings, private residences, streets, or the like. Such permanent assets may be privately or governmentally-owned. Furthermore, the geo-locations of such permanent assets may be known or may be continuously determined based on a geo-location transceiver (e.g., GPS, cellular, WiFi) associated with the permanent asset.

Embodiments of the present invention provide for such permanent assets to be connected to embodiments of the present invention via the communications network 206. As such, the permanent assets are operable to collect event data (e.g., images and videos) and upload the event data to the server devices 202 and/or stream the event data to the computing device 204 of ES users. As noted, the event data provided by the permanent assets may be classified as with the event classification of a given emergency event being undergone by a particular civilian user, such that the event data is operable to be displayed to the ES user responding to the particular emergency event. Nonetheless, it is understood that all event data, whether or not classified as part of an event classification, will be saved in the database of the server device 202 for later use, as may be required.

As such, embodiments of the present invention provide a method 1200 for capturing and presenting event data from a plurality of users. In particular, the method includes the initial Step 1202 of receiving user profile data from a computing device of a primary user and the second Step 1204 of receiving information indicative of the primary user reporting an event. A next Step 1206 includes receiving primary event data from the computing device of the primary user. Thereafter, in Step 1208, embodiments provide for the primary event data to be available for presentation on a computing device of a secondary user. The method 1200 includes additional Step 1210 of generating, based on the user profile data of the primary user, an event classification used to classify event data as being associated with the event. A next Step 1212 includes receiving a plurality of user profile data from computing devices of a plurality of tertiary users, and a next Step 1214 includes receiving a plurality of tertiary event data from the computing devices of the plurality of tertiary users. Embodiments of the present invention provide for Step 1216 to include classifying, based on the user profile data of the tertiary users, a first portion of the tertiary event data as being within the event classification. Finally, Step 1218 includes providing for the first portion of the tertiary event data to be available for presentation on the computing device of the secondary user, and a remaining portion of the tertiary event data not included within the first portion to not be available for presentation to the computing device of the secondary user. As such, embodiments of the present invention provide for the ES user to observe in real-time event data that is (1) provided by a particular civilian user undergoing an emergency event, and (2) provided by other civilian users and that may be associated with the emergency event.

In addition to reviewing event data uploaded by other civilian users, embodiments of the present invention allow for the ES user to determine the location of any such other civilian user that may be in relatively-close vicinity of the emergency event being undergone by the particular civilian user. For example, the ES user can select to view each of the civilian users that have a geo-location within a particular distance from the emergency event. In some embodiments the particular distance may be 1 block, ¼ mile, ½ mile, 1 mile, 2 miles, 5 miles, or more. In some embodiments, such other civilian user may be presented to the ES user in a list format. In alternative embodiments, the geo-location of the other civilian users may be presented via a graphic map (presented on the GUI of the ES user's computing device 204), which illustrates the geo-location of the emergency event (and thus the civilian user undergoing the event) and any other civilian users within the particular distance from the emergency event. Regardless, the ES user can select, via the GUI, one or more of the other civilian users to determine if they are providing event data or event descriptions. If the other civilian users are providing such event data or event descriptions, the ES user can select to have such event data or event descriptions presented on the computing device 204 of the ES user.

Similarly, embodiments of the present invention may provide for the geo-location of the ES user to be presented on a graphic map, along with each of the other ES user that has a geo-location within a particular distance from the emergency event. In some embodiments the particular distance may be 1 block, ¼ mile, ½ mile, 1 mile, 2 miles, 5 miles, or more. As such, the ES user can select on one or more of the other ES users to determine if they are providing event data or event descriptions. If the other ES users are providing such event data or event descriptions, the ES user can select to have such event data or event descriptions presented on the computing device 204 of the ES user.

In additional embodiments, the present invention is capable of allowing each ES users that is within the particular distance of the emergency event to establish a communications link with each other. Such a communications link may provide for such ES users to communicate in real-time with respect to the emergency event. The communications link may provide for text, voice, and/or video communications between the ES users. As such, embodiments of the present invention facilitate the communication and coordination of ES users when responding to the emergency event. For example, while communicating with other ES users via text, voice, or video communications, the ES users can also illustrate graphic instructions to the other ES users by providing instructions on a graphic map, displayed on the GUI of each of the ES users' computing device 204. In particular, the graphic map may represent a geographic area surrounding the emergency event. As such, the instructions provided on the graphic map can be used to direct each of the ES users to be strategically positioned at particular locations around the emergency event location. Because of the potential sensitive nature of the communications between the ES users, the communications link between the ES users may be encrypted, so as to provide enhanced security for the communications between ES users.

Once the ES user has responded to the emergency event, embodiments of the present invention provide for all of the information and data that was captured and/or otherwise provided by civilian users and/or ES user and that was related to the emergency event (e.g., user profile data, event descriptions, event data) to be stored for further use in the server device 202 (or the associated database). In some embodiments, such data and information may be stored in the form of reports, such as may be used by police, fire department personnel, and/or EMTs. As such, all of the information and data may is available, via embodiments of the present invention, for further use (e.g., investigations, analysis, reporting, etc.) after the emergency event has concluded.

As mentioned above, all the event data (i.e., images/videos) that was uploaded, regardless of the source, is stored in the server device 202 (or the associated database) for further use (e.g., investigations, analysis, reporting, etc.). To facilitate finding event data that may be applicable for such further use, embodiments of the present invention provide for the event data stored in the server device 202 to be associated with various event data tags that identify the event data. In certain embodiments, the event data tags may include (1) any of the information included in the user profile of a civilian user, and/or (2) any information included in the event description provided by the civilian user that was used for describing the emergency event. The event data tags may be associated with the event data via various methods of data linking, such as pointers, arrays, linked lists, or the like. As such, the event data tags will provide a link to those portions of event data which are associated with the event data tags. Embodiments will, thus, provide for users to search for specific event data by using various search terms and/or keywords. Specifically, a user can begin a query by enter a search term into a computing device 204. Embodiments of the present invention will thus compare the search term with each of the event data tags stored in the server device 202. If the search term is matched with an event data tag, embodiments of the present invention will make available for display the event data that is associated with the event data tag.

As an illustrative example, a civilian user undergoing an emergency event, such as an assault, may have captured a video of a portion of the assault. As such, the event data (i.e., the video) will be uploaded and stored in the server device 202. In addition, the civilian user may also enter an event description that describes the emergency event. For instance, the civilian user may enter the textual phrase “Yankee's t-shirt” as event description corresponding to a shirt that was worn by the assaulting party during the emergency event. As such, the phrase and/or individual terms “Yankee's t-shirt” may be used as event data tags that are linked with the event data uploaded by the civilian user. If at a later time an ES user, such as a police detective, is investigating a series of assaults being performed by an assailant commonly known to wear a “Yankee's t-shirt,” the ES user can enter the phrase “Yankee's t-shirt” into a computing device 204 as a search term in a query. As such, embodiments of the present invention will compare the search term with all of the event data tags stored in the server device 204. Once a match between the search term and the event data tag is made, the event data tag will link to the event data uploaded by the civilian user, such that the event data will be made available for review by the ES user. As such, the ES user can review the event data to potentially obtain a video of the assailant that has been committing assaults.

In additional embodiments, the event data tags may be representative of a geo-location included in the user profile data. For example, the event data tags may comprise a latitude/longitude, and address, an area code, a zip code, or other form of geo-location. As such, embodiments provide for the search term to also be representative of a geo-location, such that a user can perform a query for event data based on geo-location. For example, with both the event data tag and the search term being representative of geo-locations, the query may return a matched if the geo-location represented by the search term is within a given range from the geo-location represented by the event data tag. In some embodiments, for example, the given range may be 500 feet, ½ mile, 1 mile, 5 miles, or more.

As such, embodiments of the present invention include a method 1300 for organizing event data from a plurality of users, such that the event data is accessible for other user. The method 1300 includes the initial Step 1302 of receiving user profile data from a computing device of a first user. A next Step 1304 includes receiving event data from the computing device of the first user and storing the event data in a database. A next Step 1306 includes receiving an event description from the computing device of the first user. Embodiments of the present invention include a Step 1308 of generating, based on the user profile data and/or the event description, a set of one or more event data tags used to identify the event data stored in the database. A next Step 1310 includes receiving an indication of a second user performing a query of the database using a search term. Next, in Step 1312, a determination is made as to whether the search term matches any of the one or more event data tags. Finally, in Step 1314, upon determining that the search term matches any of the one or more event data tags, present the event data on the computing device of the second user.

As previously described, embodiment of the present invention provide for all of the information and data obtained via embodiments of the present invention to be stored in the server device 202, such that the information and data may be used for additional purposes and/or uses. In certain instances, embodiments provide for the information and data to be made available across various departments and agencies. For example, if an ES user from a particular police unit captured event data relating to a particular emergency event, such event data may be made available for access and review by other ES users that have access to embodiments of the present invention, such as ES user from other police departments, governmental agencies (e.g., ATF, FBI, CIA, etc.), or military departments (e.g., Army, Navy, Air Force, USMC, Coast Guard, etc.).

Furthermore, embodiments of the present invention provide for information and data obtained via embodiments of the present invention to be analyzed so as to determine potential trends and connections between emergency events. For example, embodiments provide for all information and data related to emergency events to be presented on a graphic map that display various geographic areas and boundaries, such as countries, states, counties, cities, towns, neighborhoods or the like. As such, users of embodiments of the present invention can graphically display all information and data related to various emergency events and overlay such information and data on a geographic map. Furthermore such graphic maps can be further refined to display only those emergency events that take place within a given time period. As an illustrative example, a user may be able to review all criminal-type emergency events that have taken place within a city within the past two weeks. As such, users of embodiments of the present invention may be able to determine which areas (e.g., a neighborhood) within a given geographic area (e.g., a city) have the highest concentration of criminal activity.

In further embodiments, the event data tags, described above with respect to the method 1300 for organizing and searching event data, may be used for analysis to determine potential trends and connections between emergency events. As previously described, the event data tags are generated based user profiles and event descriptions. As such, certain event data tags may be generated multiple times, with each generated event data tag being associated with a separate, individual emergency event. For example, and returning to method 1300, if a user preforms a query using a search term, which results in a plurality of matches to event data tags associated with a plurality of emergency events, embodiments of the present invention may be configured to provide a graphic map of the geo-location of each such emergency event. Consequently a user may be presented with a graphic map illustrating the geo-locations of each of each of the emergency events that are commonly associated with the matched event data tags. Thus, embodiments may provide for a graphic map of all emergency events of that (1) are commonly associated with a given event data tag, (2) that have taken place within a particular geographical area (e.g., city, town, etc.), and (3) have occurred within a given timeframe. For example, if a user performed a query using the search term “robbery”, embodiments of the present invention may graphically display all emergency events that are commonly linked by the “robbery” event data tag. As such, embodiments of the present invention may be capable of graphically displaying all bank robberies that occurred within the past two weeks in a particular city. Embodiments may also provide for such a graphic map to be color coded based on event descriptions (provided by civilian users and/or ES users) associated with the emergency events. For example, robbery-related emergency events (which may be associated with a “robbery” event data tag) may have a geo-location identification, such as a pin, that is colored red. Similarly, drug-related emergency events (which may be associated with a “drug” event data tag) may have a geo-location identification, such as a pin, that is colored green. As such, the user may be able to gain certain information based the graphical representation of such emergency events, as graphically illustrated and delineated via such event data tags. As such, the user may be able to gain certain information based on the concentration of the bank robbery geo-locations so as to assist with an investigation into such bank robberies.

### Additional Features

In addition to facilitating the ability for civilian users to request and obtain emergency services from ES users, embodiments of the present invention facilitate the ability of civilian user to obtain non-emergency services from non-ES users, such as other civilian users. For example, if a civilian user is undergoing a non-emergency event (e.g., a vehicle breakdown, locked keys in the vehicle, requesting transportation, or the like), the civilian user can request services from one or more other civilian users, such as a family members or friends of the civilian user. In certain embodiments, the civilian user can create a preferred set of one or more other civilian users that comprise the civilian user's preferred non-emergency contacts in the case of a non-emergency event. As described above, the preferred set of civilian users may comprise the civilian user's family members or friends. As such, in certain embodiments, when civilian user activates the present invention to request emergency services, such as illustrated in FIG. 8, the civilian user may also be provided with an option to request to receive non-emergency services from the civilian user's preferred set of other civilian users (such an option is not illustrated in FIG. 8). Upon providing an indication to request non-emergency services from the preferred set of other civilian users, the civilian user may be presented with a listing of each of the other civilian users in the civilian user's preferred set. From the listing the civilian user can select the other civilian user in the preferred set from which the civilian user wishes to request services. Upon making a selection, a communications link will be established between the civilian user and the selected other civilian user from the preferred set. As with the communications link described previously with respect to the ES user, the communications link between the civilian user and the other civilian user may be used to facilitate text, voice, and/or video communication. As such, the civilian user can request to obtain services from the other civilian user via the communications link. In situations in which the a communications link cannot be established (e.g., the other civilian user is unavailable), then the civilian user requesting the service can provide a textual, voice, or video message that is recorded in the server device 202 and that is made available for review by the other civilian user once such other civilian user is available.

Embodiments of the present invention additionally provide for the geo-locations of each of the other civilian user's included in the civilian user's preferred set to be available for presentation on the computing device 204 of the civilian user. As such, the civilian user is able to determine a precise geo-location of each of the other civilian users in the civilian user's preferred set at all times. Such an embodiment may be beneficial, for instance, in cases in which the preferred set of other civilian users comprises family members of the civilian users. As such, the civilian user can select to view the geo-location of each of the civilian user's family members at any given time. As previously described, the geo-locations of each of the other civilian users may be presented in a list-form, such as by listing the latitude/longitude, the city, the town, the area-code, the zip-code, or other similar identification of the civilian user's geo-location. In other embodiments, the civilian user will be presented with a graphical map that includes a graphical depiction of each of the geo-locations of the other civilian users in the preferred set. Such a graphical depiction may be in the form of a marker, such as a dot, a circle, a pin, or the like.

In instances in which the civilian user is undergoing an emergency event and has requested emergency services from an ES user, as previously described, embodiments of the present invention may provide for the other civilian users in the civilian user's preferred set to have access to the information and data related to the emergency event (i.e., user profile data, event descriptions, event data). As such, in addition to ES users having access to the information and data related to the emergency event, embodiments of the present invention provide for the other civilian user in the civilian user's preferred set to have access to the information and data. Thus, in instances in which the preferred set comprises family members of the civilian user undergoing the emergency event. The family members can have access to the most current information related to the civilian user and the emergency event. For example, if a civilian user is undergoing a house fire, the civilian user can make available to the civilian user's family members the event description that was provided by the civilian user. Thus, the family members can obtain current information, from the civilian user's perspective, as the causes, results, and current status of the fire. In certain instance, if ES users have responded to the emergency event and provided their own additional information and data, such additional information and data may also be made available to the other civilian users in the civilian user's preferred set.

Although the previously-detailed embodiments have been described with respect to a civilian user requesting to obtain emergency services for an emergency event being undergone by the civilian user, embodiments of the present invention provide for the civilian user to, alternatively, request emergency services for an individual other than the civilian user and/or for an emergency event not being undergone by the civilian user. For example, if a civilian user observes an individual involved in a vehicle accident, the civilian user can use embodiments of the present invention to request emergency services from an ES user for the individual. Such features may be facilitated by prompting the civilian user whether the civilian user's request is for the civilian user or for an other individual. The prompting may be provided before or simultaneously with the civilian user reporting an event and requesting emergency services, such as in the GUI illustrated in FIG. 8. If the civilian user indicates that the request is for the civilian user, then embodiments proceed as previously described. If the user indicates that the request is for an other individual, the civilian user may be further requested to provide an indication of the emergency event type (e.g., criminal, fire, medical, etc.) and to provide any applicable event descriptions. Furthermore, if the civilian user cannot remain in the geo-location where the individual is undergoing the emergency event. The civilian user can provide the geo-location of the individual (i.e., lat/long, address, street intersection, etc.) of the individual. In other embodiments, the civilian user can drop a geo-location marker on a graphic map, with such marker indicating the geo-location of the individual. As such, a responding ES user will be provided with the geo-location of the individual, such that the ES user can efficiently reach the individual so as to provide emergency services.

Additionally, embodiments of the present invention may provide for the creation of an emergency event resource application that allows civilian users to obtain and exchange information related to a particular emergency event. In more detail, for certain emergency events, embodiments of the present invention may provide a forum on which civilian users can provide information and data that can be shared with other civilian users, such that the other civilian users can be apprised and updated as to the emergency situation. In some embodiments, the event resource application will be automatically generated for a particular type of emergency event. For example, if a large-scale, criminal-type emergency event, such as a terrorist attack (as determined by event description and/or event data submitted by a civilian user), is underway, embodiments of the present invention may automatically generate an event resource application applicable to the emergency event. As such, any civilian user can access the event resource application, so as to provide and obtain information and data related to the event.

In more detail, the event resource application may include a “chat-room” feature by which civilian users can provide real-time information and data (e.g., textual updates) regarding the emergency event. In other embodiments, the event resource application may provide for an audio and/or video communications link between participants of the event resource application, such that the civilian users can conduct live voice and/or video communications. In some embodiments, the civilian user can also upload event data (i.e., images/videos) of the emergency event for review by other civilian users. In some situations, there may be multiple large-scale emergency events occurring simultaneously, such as a terrorist attack and a building fire. As such, embodiments may provide for multiple event resource applications to be available simultaneously. In such instances, embodiments of the present invention provide for civilian users to participate in each event resource application simultaneously, such as via a “split-screen” on the GUI of the civilian users' computing devices 204. In other embodiments, the civilian users can alternate back and forth between event resource applications. The event resource application may also provide a graphic map that illustrates the geo-location of the emergency event and any ES users and civilian users within a particular distance from the emergency event. As such, a civilian user accessing in the event resource application can select on one or more of the users presented on the graphic map to determine if they are providing event data or event descriptions. If such users are providing such event data or event descriptions, the civilian user participating in the event resource application can select to have such event data or event descriptions presented on the computing device 204 of the civilian user.

Embodiments of the present invention additionally provide for users (including civilian and ES users) to be presented with information related to the geo-location of medical service facilities available to provide medical aid to users. Such medical service facilities may be, for instance, hospitals, doctor's offices, police/fire stations, first-aid stations, or the like. Embodiments may present the geo-locations of such medical service facilitates based on various criteria. For example, a user can determine the closest medical service facility to a geo-location of the user. Alternatively, the user can determine each of the medical service facilities within a given distance from the geo-location of the user. In further alternatives, the user can determine each of the medical facilities within a provided geographic area (e.g., city, county, neighborhood, area-code, zip-code, etc.). In some embodiments, the medical service facilities may be presented in list form. However, in other embodiments, the user may be provided with a graphic map, via the GUI of the user's computing device 204, which graphically illustrates the geo-location of the medical service facilities (and the geo-location of the user, if applicable). In certain additional embodiments, the present invention is operable to provide directions, either step-by-step textual directions or graphical route directions, from the geo-location of the user to one or more of the medical services facilities. In addition to medical facilities, embodiments of the present invention may store geo-locations and/or contact information (e.g., address, email, website) of a plurality of other services, such as emergency food supplies, emergency water supplies, emergency clothing supplies, temporary/permanent shelters and housing, schools, jobs, and the like.

Embodiments of the present invention additionally provide a first-aid resource for allowing users to access and obtain first-aid information. For example, the server device 202 may include first-aid manuals in the form of textual instructions, audio instructions, video instructions, or the like, with each being available for presentation on the computing devices 204 of users. The first-aid manuals may include information on treating various medical-type emergency events, such as, for instance, burns, hypothermia, snake-bites, gunshots wounds, stabbings, shock trauma, or any other type of medical-type emergency event. In certain embodiments, the first-aid manuals may be listed alphabetically by emergency event to facilitate searching. In other embodiment, the user may search all available first-aid manuals to find the manual appropriate for the user's emergency event.

Embodiments of the present invention additionally allow for users to donate monetary amounts to a plurality of charitable organizations. In more detail, charitable organizations can register, via embodiments of the present invention, to have their respected organizations/charities presented to users (either civilian users or ES users) for purposes of obtaining donations. As such, if a user wishes to provide a donation to a particular charitable organization, the user can select the particular charitable organization and submit a donation. Embodiments provide for the donation to be made via a “check-out” process, in which the user selects (1) a monetary amount that the user wishes to donate, and (2) payment information. In certain embodiments, the present invention will automatically obtain payment information from the user's financial information (e.g., credit card information, bank account information, etc.) included in the user's user profile. Once the donation has been made, embodiments of the present invention will generate a receipt, which may be electronic or printable, and which the user may use for deduction purposes when preparing the user's taxes.

Embodiments of the present invention are also operable to provide users with referrals to insurance providers, attorneys, and medical service providers. Such insurance providers, attorneys, and medical service providers that wish to be referred to users may register, via embodiments of the present invention. Embodiments of the present invention provide for insurance providers, attorneys, and medical service providers to be automatically presented to certain users for recommendations. For example, if while providing user profile information, a user indicated that the user did not have a certain type of insurance, then embodiments of the present invention would recommend that the user obtain such insurance by presenting one or more recommended insurance providers. For example, if a user indicated that the user did not have health insurance, embodiments of the present invention may present one or more recommended health insurance providers from which the user could obtain health insurance. Similar recommendations may be made, in instances where the user indicated that the user did not have residential or vehicle insurance. Embodiments of the present invention will facilitate the transfer of information to the insurance providers, such that the user can quickly and efficiently obtain an insurance policy. For example, embodiments of the present invention can transfer portions of the user's user profile information necessary for obtaining and underwriting an insurance policy form the insurance provider. In still further embodiments of the present invention, the user is able to provide information to the insurance provider so as to facilitate the submission of an insurance claim. For example, if the user is in a vehicle accident (i.e., an emergency event) and the user obtained and uploaded event data in the form of digital images of the damaged vehicle, embodiments of the present invention provide for such event data to be transmitted to the insurance provider and/or appraiser/claims adjuster, so as to facilitate the insurance providers ability to reimburse the user for the insurance claim. Additional embodiments may also provide for the user to pay for the insurance provider services automatically by providing payment information directly to the insurance providers. As previously described, the payment information may comprising the user's financial information (e.g., credit card, bank account, etc.) included in the user's user profile.

Similarly, embodiments of the present information may automatically present attorneys for recommendation to certain users. For example, if a user was involved in an emergency event that may require future legal representation (e.g., vehicle accident, workplace accident, etc.), embodiments of the present invention may provide a recommendation of one or more attorneys that are skilled in the necessary practice areas to provide legal representation to the user. As with the insurance providers, embodiments of the present invention facilitate the transfer of the user's user profile information to a selected attorney so as to facilitate engagement of the attorney's services. Additional embodiments may also provide for the user to pay for the attorney services automatically by providing payment information to the attorney. As previously described, the payment information may comprising the user's financial information (e.g., credit card, bank account, etc.) included in the user's user profile.

Furthermore, embodiments of the present information may automatically present medical service providers (e.g., doctors, dentists, chiropractors, physician assistants, etc.) for recommendation to certain users. For example, if a user was involved in an emergency event that may require the use to obtain emergency services (e.g., vehicle accident, workplace accident, etc.), embodiments of the present invention may provide a recommendation of one or more medical service providers that are skilled in the necessary practice areas to provide required medical services to the user. Embodiments of the present invention facilitate the transfer of the user's user profile information to a selected medical service provider to facilitate engagement of the medical service provider. In addition to simply providing for user profile data to be transferred to medical service providers, embodiments of the present invention are operable to act as a repository for the user's medical information and medical history. For example, when a user becomes a patient of a new medical services provider, the user's user profile information, including personal, health, and insurance information, may be transferred to the new medical service provider such that the user does not have to manually provide such information to the new medical services provider. In some embodiments, the user will be required to verify the user's identity and intent to transfer such information by re-entering the user's username and password, answering the security questions, or other similar method of verification/authentication. Additional embodiments may also provide for the user to pay for the medical services automatically by providing payment information to the medical service provider. As previously described, the payment information may comprising the user's financial information (e.g., credit card, bank account, etc.) included in the user's user profile.

Furthermore, once the new medical services provider has performed service for the user, any heath records pertaining to the services (e.g., doctor notes, test results, x-rays, scans, or the like) may be uploaded and stored in the server device 202. As such, when the user is required to see any other medical service provider, the user's user profile information along with the health records performed by the new medical services provider will be available for review by the any such other medical service provider. Thus, embodiments of the present invention are able to operate as a medical record repository, by facilitating the storage and transfer of the user's medical records. In such embodiments, the server device 202 and the computing devices 204 will be equipped to provide secure communications and data storage in compliance with Health Insurance Portability and Accountability Act (HIPPA). For instance, such secure communications and data storage may include encrypted communications and data storage.

Although the invention has been described with reference to the embodiments illustrated in the attached drawing figures, it is noted that equivalents may be employed and substitutions made herein without departing from the scope of the invention as recited in the claims.

Having thus described various embodiments of the invention, what is claimed as new and desired to be protected by Letters Patent includes the following:

1. A non-transitory computer-readable storage medium with a computer program stored thereon for capturing data from a plurality of users, wherein the computer program instructs a processor to perform the following steps:
receive user profile data from a computing device of a primary user;
receive information indicative of the primary user reporting an event;
receive primary event data from the computing device of the primary user;
provide for the primary event data to be available for presentation on a computing device of a secondary user;
generate, based on the user profile data of the primary user, an event classification used to classify event data as being associated with the event;
receive a plurality of user profile data from computing devices of a plurality of tertiary users;
receive a plurality of tertiary event data from the computing devices of the plurality of tertiary users;
classify, based on the user profile data of the tertiary users, a first portion of the tertiary event data as being within the event classification; and
provide for the first portion of the tertiary event data to be available for presentation on the computing device of the secondary user;
wherein a remaining portion of the tertiary event data not included within the first portion is not available for presentation to the computing device of the secondary user.

2. The computer-readable storage medium of claim 1, wherein the user profile data comprises: a geo-location of the user, a home address of the user, or an area code of a telephone number associated with the user.

3. The computer-readable storage medium of claim 2, wherein the user profile data comprises a geo-location of the user, and wherein the geo-location of the user is obtained from one or more of the following: a cellular network, GPS, a WiFi network, and a Bluetooth network.

4. The computer-readable storage medium of claim 1, wherein the primary and tertiary event data are selected from one or more of the following: video data, image data, audio data, and textual data.

5. The computer-readable storage medium of claim 1, wherein the event classification is generated based on a geographic area having a boundary within a given range from a geo-location of the primary user.

6. The computer-readable storage medium of claim 1, wherein the computer program instructions the processor to perform the following additional steps:
receive permanent asset profile data from a computing device associated with a permanent asset;
receive permanent asset event data from the computing device associated with the permanent asset;
determine, based on the permanent asset profile data of the permanent asset, whether the permanent asset event data is within the event classification; and
if the permanent asset event data is within the event classification, provide for the permanent asset event data to be available for presentation on the computing device of the secondary user.

7. The computer-readable storage medium of claim 6, wherein the permanent asset is a security system having one or more security cameras.

8. The computer-readable storage medium of claim 1, wherein the step of the primary user reporting an event comprises receiving an automated alert originating from an electronic device associated with the primary user.

9. The computer-readable storage medium of claim 8, wherein the electronic device is selected from one or more of the following: a pacemaker, a heart-rate monitor, a blood-pressure monitor, a home security system, or a fire detection system.

10. A non-transitory computer-readable storage medium with a computer program stored thereon for organizing data from a plurality of users, wherein the computer program instructs a processor to perform the following steps:
receive user profile data from a computing device of a first user;
receive event data from the computing device of the first user and store the event data in a database;
receive an event description from the computing device of the first user;
generate, based on the user profile data and/or the event description, a set of one or more event data tags used to identify the event data stored in the database;
receive an indication of a second user performing a query of the database using a search term;
determine whether the search term matches any of the one or more event data tags; and
upon determining that the search term matches any of the one or more event data tags, present the event data on the computing device of the second user.

11. The computer-readable storage medium of claim 10, wherein the user profile data and/or the event description comprises: a geo-location of the user, a home address of the user, or a date/time stamp.

12. The computer-readable storage medium of claim 10, wherein the event data is selected from one or more of the following: video data, image data, and audio data.

13. The computer-readable storage medium of claim 10, wherein the event data tag is a keyword included in the event description.

14. The computer-readable storage medium of claim 10, wherein the event data tag is representative of a geo-location included in the user profile data.

15. The computer-readable storage medium of claim 14, wherein the search term represents a geo-location, and wherein the geo-location represented by the search term is within a given range from the geo-location represented by the event data tag.

16. A non-transitory computer-readable storage medium with a computer program stored thereon for generating a communication link between a civilian user and one or more emergency service users, wherein the computer program instructs one or more processors to perform the following steps:
receive user profile data from a plurality of emergency service users, wherein the user profile data indicates whether each emergency service user in the plurality is an emergency service user of a first type or a second type;
provide a set of computer-executable instructions that, when executed by a computing device of the civilian user, generates a user interface displayable on an electronic display coupled to the civilian user's computing device;
present, via the user interface of the civilian user's computing device, a solicitation for the civilian user to select an emergency event type from a plurality of event types;
receive information indicative of the civilian user selecting a first emergency event type;
based on a comparison between the first emergency event type and the user profile data of the emergency service users, send a notification to an emergency service user of either the first type or the second type, wherein the emergency service user to which the notification is sent is a first emergency service user;
provide a set of computer-executable instructions that, when executed by a computing device of the first emergency service user, generates a user interface displayable on an electronic display coupled to the first emergency service user's computing device;
present, via the user interface of the first emergency service user's computing device, a solicitation for the first emergency service user to communicate with the civilian user; and
upon receiving information indicative of an assent to communicate with the civilian user, open a communication link between the first emergency service user and the civilian user, via the computing devices of the first emergency service user and the civilian user.

17. The computer-readable storage medium of claim 16, wherein first type of emergency service user is selected from: a police officer, a fireman, an emergency medical technician, or a second civilian user.

18. The computer-readable storage medium of claim 17, wherein the second type of emergency service user is selected from a police officer, a fireman, an emergency medical technician, or a second civilian user, wherein the second type of emergency service user is different from the first type of emergency service user.

19. The computer-readable storage medium of claim 16, wherein the emergency event type is selected from: a criminal emergency, a fire emergency, or a medical emergency.

20. The computer-readable storage medium of claim 16, wherein the communication link is selected from: an audio-telephone link, a video-telephone link, or a text message link.
